(19) United States
(12) Patent Application Publication
(10) Pub. No.: US 2024/0197221 A1
(43) Pub. Date: Jun. 20, 2024

(54) FRAGRANCE FOR IMPROVING HAPPINESS STATE AND METHOD OF ASSESSING

(71) Applicant: GIVAUDAN SA, VERNIER (CH)

(72) Inventors: Giuliano GAETA, Ramsgate Kent (GB); Alan Forbes PROVAN, Kingsnorth, Ashford Kent (GB); Natalie Anuradha T.D. GUNASEKARA, Pinner, Greater London (GB)

(21) Appl. No.: 18/555,712

(22) PCT Filed: May 30, 2022
(86) PCT No.: PCT/EP2022/064553

§ 371 Date: Oct. 16, 2023

(30) Foreign Application Priority Data
May 28, 2021 (GB) 2107716.9

(51) Int. Cl. A61B 5/16; A61B 5/00; A61B 5/1455; A61M 21/00; A61M 21/02; C11B 9/00

(52) U.S. Cl. CPC A61B 5/165; A61B 5/0075; A61B 5/14553; A61B 5/4836; A61M 21/02; C11B 9/0061; A61M 2021/0016

(57) ABSTRACT

The present disclosure relates to methods of assessing the ability of a test fragrance ingredient or a test fragrance composition to improve the happiness state of a human subject and of creating fragrance compositions having a positive happy effect on a human subject. It further relates to fragrance compositions for improving the happiness state of a human subject, to consumer products comprising such fragrance compositions, and to methods of improving the happiness state of a human subject.

The present invention relates to methods of assessing the ability of a test fragrance ingredient or a test fragrance composition to improve the happiness state of a human subject and of creating fragrance compositions having an effect of improving the happiness of a human subject. It further relates to fragrance compositions for improving the happiness state of a human subject, to consumer products comprising such fragrance compositions, and to methods of improving the happiness state of a human subject.

Perfumery has been widely employed by consumer product companies in order to impart to their products pleasant, well-liked odours that promote consumer liking and which influence purchasing decisions for this reason.

However, in an increasingly competitive marketplace, mere liking is often not sufficient to differentiate one brand over its competitors. Accordingly, in the market execution of their products, consumer product companies frequently refer to wide-ranging product benefits, typically communicated through diverse advertising campaigns, as well as on the packaging and labelling of their products, which together form an important part of their branding strategy. New differentiating effects are constantly sought, and perfumery has often been employed as a means to achieve those effects. For example, perfumery has been employed to create real or perceived functional effects that may relate to cosmetic effects, hygiene effects, malodour-counteracting effects, and the like.

It has long been known that fragrance materials and essential oils can promote feelings of happiness and well-being. These materials have also been used in cosmetic products and aromatherapy in order to provide a similar effect.

Aroma-Chology® is a term coined by the Olfactory Research Fund Ltd. (see extensive review by J. Jellinek in Cosmetics & Toiletries, (1994) 109, pp 83-101). It is concerned specifically with the temporary, beneficial psychological effects of aromas in human behaviours and emotions to improve mood and quality of life. In fact, a large number of products promoted as having aromatherapy benefits can be more accurately identified for their Aroma-Chology® benefits as they produce temporary psychological effects. However, there is no teaching as to how to formulate products to achieve such benefits qualitatively or quantitatively with a reliable expectation of success. In addition, it is well known that fragrances can be perceived as associated with different attributes in different countries.

More recently, several patent applications (e.g. WO 02/49600, WO 2008/050084, WO 2008/050086, WO 2020/165463) have focused on providing positive mood benefits through fragrance compositions, providing guidelines on how to measure these mood benefits and on how to create effective fragrance compositions.

For instance, WO 2008/050086 describes fragrance compositions, which deliver well-being benefits through frequent, low level positive mood stimulation, and in particular happiness, to a human subject. WO 2008/050086 provides formulation guidelines specifying different types of fragrance ingredients (e.g. happy, relaxing or neutral ingredients) and the concentrations at which these fragrance ingredients may be present in the compositions in order to achieve the desired effect. These formulation guidelines have been developed based on consumer testing.

WO 2008/050086 classifies fragrance ingredients into six categories: HMP fragrance ingredients that are strongly associated with happy moods; HMR fragrance ingredients that may support both happy and relaxing moods; HMI fragrance ingredients that may support both happy and invigorating moods; RMP fragrance ingredients that strongly support relaxing moods; IMP fragrance ingredients that strongly support invigorating moods; and GEN fragrance ingredients that may support a variety of moods.

However, there is a large group of other fragrance ingredients that have not been assigned to any of these three groups. Also, some fragrance ingredients are now longer used nowadays, either due to regulatory restrictions or due to the availability of better alternatives.

There is therefore a need to classify further fragrance ingredients.

WO 2008/050086 uses consumer testing for assessing the influence of a fragrance composition on the mood and emotions of test subjects. However, consumer testing has several important drawbacks:
Lack of any physiological information from the participant
No information on implicit mental processes happening in the brain
Explicit/declarative description of odours is much harder than other stimuli (e.g. the description of a picture), often resulting in inaccurate findings
Participants are able to control the answers provided to a test and might alter their answers to please the experimenter (a phenomenon known in the psychology literature as “participant bias”), leading to the collection of inaccurate data (https://doi.org/10.1016/0191-8869(86)90014-0)
Difficulty to differentiate different mood states between samples
Ratings often correlate with liking scores
Tendency to acquiesce—answer positively
Tendency to be evasive—to opt for neutral, unsure, uncertain response
Tendency for speed—to complete the test quickly rather than accurately

It is therefore highly desirable that an improved technique for assessing the mood state of a human subject is developed, which allows for more flexibility during the measurement.

The above problems are solved by the present invention.

In a first aspect, the present invention provides a method of assessing the happiness state of a human subject by means of fNIRS (functional near-infrared spectroscopy).

In a second aspect, the present invention provides a method of assessing the ability of a test fragrance ingredient or a test fragrance composition to improve the happiness state of a human subject.

In a third aspect, the present invention provides a method of creating a fragrance composition having an effect of improving the happiness of a human subject.

In a fourth aspect, the present invention provides a fragrance composition for improving the happiness state of a human subject.

In a fifth aspect, the present invention provides a consumer product comprising said fragrance composition.

In a sixth aspect, the present invention provides a method of improving the happiness state of a human subject, comprising the step of providing an effective amount of the fragrance composition of the invention to the human subject.

In a seventh aspect, the present invention relates to the use of certain fragrance ingredients for improving the happiness state of a human subject.

The use of functional near-infrared spectroscopy (fNIRS) for assessing the mood state, and in particular the happiness state, of a human subject is highly advantageous: fNIRS is harmless, tolerant to bodily movements, and highly portable; it is also suitable for all possible participant populations, from newborns to the elderly, and experimental settings, both inside and outside the laboratory (for a review, see: “The present and future use of functional near-infrared spectroscopy (fNIRS) for cognitive neuroscience”, Pinti et al., Ann. N.Y. Acad. Sci. 1464 (2020) 5-29). In particular, the use of fNIRS allows for in-context-testing, where participants are asked to perform specific tasks related to the fragrances provided to them (e.g. cleaning a hard surface while smelling the fragrance of an all-purpose cleaner).

fNIRS is an optical, non-invasive neuroimaging technique that allows the measurement of brain tissue concentration changes of oxygenated (Oxy Hb or HbO₂) and deoxygenated (Deoxy Hb or HbR) haemoglobin following neuronal activation. This is achieved by shining NIR light (650-950 nm) into the head, and, taking advantage of the relative transparency of the biological tissue within this NIR optical window, light will reach the brain tissue. The most dominant and physiological-dependent absorbing chromophore within the NIR optical window is haemoglobin. Based on its saturation state, we can have haemoglobin in its oxygenated (HbO₂) and deoxygenated form (HbR). In particular, HbO₂ and HbR absorb the NIR light differently: HbO₂ absorption is higher for λ>800 nm; on the contrary, HbR absorption coefficient is higher for λ<800 nm.

When a brain area is active and involved in the execution of a certain task, the brain's metabolic demand for oxygen and glucose increases, leading to an oversupply in regional cerebral blood flow (CBF) to meet the increased metabolic demand of the brain. The oversupply in regional CBF produces an increase in HbO₂ and a decrease in HbR concentrations; these are estimated by changes in light attenuation that can be measured by fNIRS.

The portion of tissue interrogated by the NIR light is called a channel and is located at the midpoint between the source optode (S) and the detector optode (D), and at a depth of around the half of the source-detector separation. To fully exploit the potential of fNIRS, multi-channel devices are used nowadays. These allow monitoring of larger portions of the head and the gathering of topographic HbO₂ and HbR maps. Several multi-channel fNIRS devices are commercially available (e.g. Brite by Artinis, ETG-4100 by Hitachi or NIRSPort by NIRx).

The position of the fNIRS channels is generally standardized based on the EEG's 10-20 system. Typical devices use about sixteen to twenty-two channels. Optodes (detectors and sources) must be placed in an alternate fashion (i.e. a source followed by a detector, followed by a source . . . ) typically in a grid with equal distances between the channels, e.g. at a distance of 3 cm (Pinti et al. (2019) “Current Status and Issues Regarding Pre-processing of fNIRS Neuroimaging Data: An Investigation of Diverse Signal Filtering Methods Within a General Linear Model Framework”, Front. Hum. Neurosci. 12:505.). Both optodes and channels are typically numbered to allow for identification. For optodes, the letter S before the number typically defines a source optode, while the D letter before the number defines a detector optode. The numbers are usually progressive, e.g. from 1 to 8 for the sources and from 1 to 7 for the detectors.

In the methods of the present invention, the following set-up of the fNIRS channels was used:

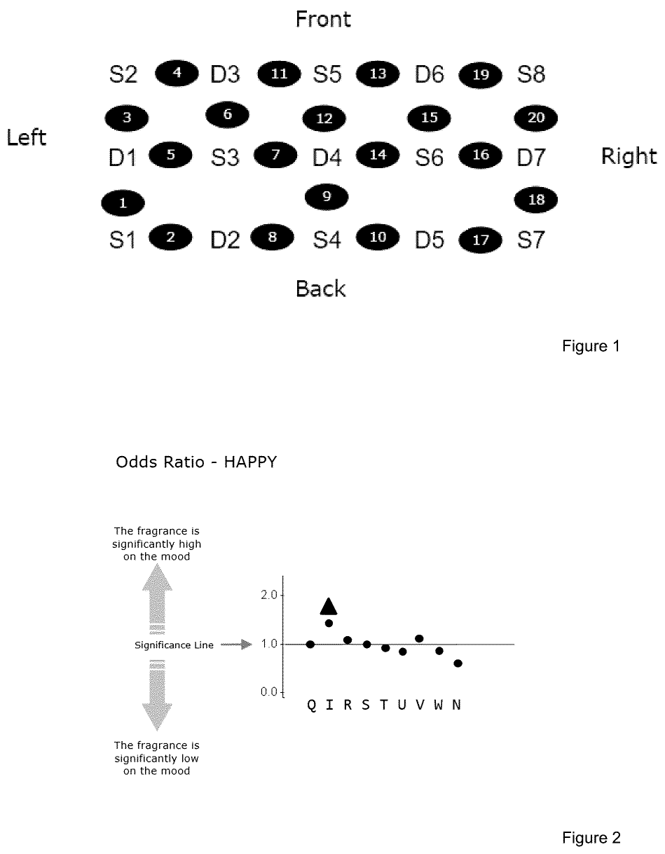

The centre of fNIRS Channel 12 was placed on the standard position EEG channel FPz according to the EEG 10-20 system (Trambaiolli et al. “Predicting affective valence using cortical hemodynamic signals”, Sci Rep 8, 5406 (2018)). Channel 12 is located between source S5 and detector D4, where S5 is situated 1.5 cm from the location of the EEG channel FPz towards the Nasion on the midline of the head, and D4 is situated 1.5 cm from the location of EEG channel FPz towards the Inion on the midline of the head. All fNIRS optodes are placed at a standardised distance of 3 cm one from one another and are arranged on gridlines extending parallel and orthogonally to the midline. Taking S5 and D4 as a reference, and considering a shift of 3 cm for each optode either on the Nasion-Inion direction (where “in front” means towards the Nasion and “behind” means towards the Inion) or on the Pre Auricolar line (where “to the left” means towards the Left Pre Auricolar line and “to the right” means towards the Right Pre Auricolar Line), then S4 is behind D4, D5 on the right of S4, S7 on the right of D5, D7 in front of S7, S8 in front of D7, S6 on the left of D7, D6 on the left of S8, D2 on the left of S4, S3 on the left of D4, D3 in front of S3, S1 on the left of D2, D1 in front of S1 and S2 in front of D1. This setup is also shown in FIG. 1.

The channel scheme is the following:

| Channel | Position |
|---|---|
| Number | (Source - Detector) |
| Channel 1 | S1 - D1 |
| Channel 2 | S1 - D2 |
| Channel 3 | S2 - D1 |
| Channel 4 | S2 - D3 |
| Channel 5 | S3 - D1 |
| Channel 6 | S3 - D3 |
| Channel 7 | S3 - D4 |
| Channel 8 | S4 - D2 |
| Channel 9 | S4 - D4 |
| Channel 10 | S4 - D5 |
| Channel 11 | S5 - D3 |
| Channel 12 | S5 - D4 |
| Channel 13 | S5 - D6 |
| Channel 14 | S6 - D4 |
| Channel 15 | S6 - D6 |
| Channel 16 | S6 - D7 |
| Channel 17 | S7 - D5 |
| Channel 18 | S7 - D7 |
| Channel 19 | S8 - D6 |
| Channel 20 | S8 - D7 |

Thus, there are nine channels per hemisphere (left or right) and two channels at the midline of the frontal and prefrontal areas. Channels 1 to 8 and 11 are located in the left hemisphere, Channels 9 and 12 are on the midline, and Channels 10 and 13 to 20 are in the right hemisphere. Channels 9 and 12 are only considered for full brain analysis.

By means of extensive research, it has been found that certain areas of the brain, and in particular certain channels, can be used as indicators for assessing the happiness state of a human subject. More specifically, an increase or decrease of Oxy Hb, Deoxy Hb and/or Total Hb (corresponding to the sum of Oxy Hb plus Deoxy Hb) in the left or right hemisphere, the full brain, or certain specific channels, at certain time points provides an indication as to whether the happiness state of the human subject is increased or decreased or stays about the same. The details will be described in relation to the method outlined below, but equally apply to the general method of assessing the happiness state of a human subject.

The above finding has been applied in the present invention to provide a method of assessing the ability of a test fragrance ingredient or a test fragrance composition to improve the happiness state of a human subject.

Said method comprises the following steps:
a) measuring a base happiness state of one or more human test subject(s);
b) providing the test fragrance ingredient or the test fragrance composition to the human test subject(s) for smelling;
c) measuring a resulting happiness state of the human test subject(s); and
d) determining a difference between the resulting happiness state and the base happiness state for the human test subject(s).

The base happiness state and the resulting happiness state are measured by functional Near Infrared Spectroscopy (fNIRS) of the human test subject(s)′ left brain hemisphere, right brain hemisphere, and full brain.

More specifically, the applicant has identified certain specific Channels, haemoglobin types and time points that are particularly indicative of the effect on the happiness state of the human subject.

In order to measure the base happiness state, the human test subject(s) may be provided with a non-fragranced sample, e.g. a piece of cotton or cloth or a sorbarod. A sorbarod is a small plastic pot containing a polyester absorbent fibre insert encased in polyethylene sleeve. The fragrance can be applied to the insert, which provides continuous refreshment of the fragrance over several assessments, and can be easily perceived when held close to the nose to smell.

Alternatively, it is also possible to measure a reference happiness state, e.g. in the presence of a reference fragrance sample.

If more than one human test subjects are involved, results for the base happiness state and the resulting happiness state may be averaged prior to determining the difference. Alternatively, it is also possible to determine the difference for each human test subject separately.

It has been found that the test fragrance ingredient or the test fragrance composition is able to improve the happiness state of the human subject if at least one of Criterion A and Criterion B is met.

Criterion A requires that at least three out of the following six conditions A1 through A6 are met:
A1. Deoxy Hb for the full brain shows a statistically significant decrease after 30 seconds of smelling;
A2. Deoxy Hb for the right brain hemisphere shows a statistically significant decrease after 30 seconds of smelling;
A3. Total Hb for the full brain shows a statistically significant decrease after 30 seconds of smelling;
A4. Total Hb for the right brain hemisphere shows a statistically significant decrease after 30 seconds of smelling;
A5. Deoxy Hb for the right brain hemisphere shows a statistically significant increase after 0-10 seconds of smelling;
A6. Oxy Hb for the right brain hemisphere shows a statistically significant increase after 5-10 seconds of smelling.

As outlined above, Total Hb is the amount of total haemoglobin measured, Oxy Hb is the amount of oxygenated haemoglobin measured, and Deoxy Hb is the amount of deoxygenated haemoglobin measured.

Haemoglobin values for the left brain hemisphere correspond to the mathematical average of the individual haemoglobin values of Channels 1 to 8 and 11, as defined above.

Haemoglobin values for the right brain hemisphere correspond to the mathematical average of the individual haemoglobin values of Channels 10 and 13 to 20, as defined above.

Haemoglobin values for the full brain correspond to the mathematical average of the individual haemoglobin values of all Channels 1 to 20, as defined above.

The effect on haemoglobin levels (Total Hb, Oxy Hb, and Deoxy Hb) may vary over time. It was found that more accurate results can be obtained by analysing haemoglobin values for several different time periods, e.g. after 0-5 seconds, 0-10 seconds, 5-10 seconds, 10-15 seconds, 15-20 seconds, 10-20 seconds, 20-25 seconds, 25-30 seconds, or after 30 seconds. Interestingly, the 5-second blocks roughly correspond to the time of a full respiration cycle (inhalation+exhalation).

In general, when it comes to the definition of brain signatures for happy fragrances and considering the average activity of larger portions of the brain, longer time intervals (and even the analysis of the full 30 seconds of exposure to the fragrance) appear to be the most relevant. This is in contrast to what was found when assessing relaxing or invigorating fragrances, where shorter time intervals appear pivotal to define the associated brain signatures.

Also, the right hemisphere appears to be more involved than the left one in the processing of happy fragrances.

At the single channel level, specific brain signatures for happy fragrances can be defined in shorter/earlier time windows, with a more balanced impact of OxyHb, DeoxyHb and TotalHb, and a higher involvement of the left hemisphere, and in particular, of channel 4.

Statistical significance is verified using a 2-tailed Student's t-test with a statistical significance threshold at 0.05.

Criterion B requires that at least five out of the following ten conditions B1 through B10 are met:
B1. Channel 3 shows a statistically significant increase of Deoxy Hb after 30 seconds of smelling;
B2. Channel 20 shows a statistically significant increase of Deoxy Hb after 30 seconds of smelling;
B3. Channel 12 shows a statistically significant decrease of Total Hb after 30 seconds of smelling;
B4. Channel 5 shows a statistically significant increase of Oxy Hb after 0-5 seconds of smelling;
B5. Channel 4 shows a statistically significant increase of Total Hb after 0-5 seconds of smelling;
B6. Channel 5 shows a statistically significant increase of Total Hb after 0-5 seconds of smelling;
B7. Channel 11 shows a statistically significant increase of Oxy Hb after 0-10 seconds of smelling;
B8. Channel 8 shows a statistically significant decrease of Deoxy Hb after 5-10 seconds of smelling;
B9. Channel 10 shows a statistically significant decrease of Deoxy Hb after 5-10 seconds of smelling;
B10. Channel 18 shows a statistically significant increase of Oxy Hb after 5-10 seconds of smelling.

As outlined above, Channels 1 to 8 and 11 are located in the left brain hemisphere, Channels 9 and 12 are located on the midline, and Channels 10 and 13 to 20 are located in the right brain hemisphere.

Throughout this application, the terms “improving the happiness state”, “increasing the happiness state”, “enhancing the happiness state”, and “increasing happiness” are used interchangeably. They are meant to express that a certain item, in particular a fragrance ingredient or fragrance composition or consumer product containing the same, has a positive mood enhancing effect on a human subject. In other words, they induce positive moods and emotions, such as happiness, and make people feel happy, uplifted, delighted, optimistic, and enthusiastic.

This emotional territory has been defined typically by the model circumplex of affect where emotions were classified in terms of their level of valence (i.e. pleasantness) and arousal (i.e. degree of activation) involved (Posner J, Russell J A, Peterson B S. The circumplex model of affect: an integrative approach to affective neuroscience, cognitive development, and psychopathology. Dev Psychopathol. 2005; 17(3): 715-734. doi: 10.1017/S0954579405050340), with happy positive moods represented by feelings of happiness, excitement and contentedness.

This emotional space has now been found to further include positive emotions, such as optimism and fulfilment, to reflect those inner feelings of happiness, and also highly activated emotions, such as delight, enthusiasm, euphoria and playfulness often shared with others.

Thus, the present application in general relates to the enhancement of positive emotions spanning the range of happiness: from positive low activated mood states, such as contentedness, to highly activated positive happy moods, such as excitement. These positive mood states of happiness include, but are not limited to, those inner feelings of happiness, such as optimism and fulfilment, and those highly activated feelings of happiness, such as delight, enthusiasm, euphoria and playfulness.

Throughout this application, the terms “fragrance” and “perfume” are used interchangeably.

Furthermore, also the terms “(fragrance) ingredient” and “(fragrance) material” are used interchangeably. In the context of the present invention, the term “fragrance ingredient” refers to an ingredient that has the function of providing a noticeable and identifiable odour to the fragrance composition. Fragrance ingredients include highly performing ingredients intended for providing an intense olfactive impression, as well as less performing ingredients intended for providing a subtle olfactive impression.

The term “fragrance composition” relates to a mixture of two or more fragrance ingredients. It may optionally include one or more odourless or low-odour solvents and/or diluents, e.g. as a vehicle for a fragrance material.

Throughout this application, the terms “(human) test subjects” and “participants” are used interchangeably.

Preferably, several human test subjects are involved in the method of the invention, in order to get a more representative and reliable result, for example more than five, more than ten, more than 15, or even more. Results from several human test subjects may be averaged. Alternatively, they may also be summed up.

Furthermore, participants that indicate that they dislike a certain test fragrance ingredient or test fragrance composition may be excluded from the respective analysis.

The method of the present invention allows for a fast, simple and reliable assessment of the ability of a test fragrance ingredient or a test fragrance composition to improve the happiness state of a human subject. Fragrances may be tested in wide variety of settings, from a non-motion laboratory setting to in-context-testing. Furthermore, the method allows for detecting sub-conscious effects, thereby avoiding common issues of conscious methods (e.g. interrogation), which often provide only limited and often inaccurate information due to dishonest responses, prior survey biases, and inarticulacy, for instance.

In order to qualify as a happy fragrance ingredient or fragrance composition, a test fragrance ingredient/composition must meet at least one of Criterion A and Criterion B. Preferably, both Criterion A and Criterion B are met.

More specifically, the applicant has identified certain specific Channels, haemoglobin types and time points that are particularly indicative of the effect on the happiness state of the human subject.

Therefore, in an embodiment, further Criterion C is met.

Criterion C requires that at least ten out of the following 20 conditions B1 through B10 and C1 through C10 are met:
B1. Channel 3 shows a statistically significant increase of Deoxy Hb after 30 seconds of smelling;
B2. Channel 20 shows a statistically significant increase of Deoxy Hb after 30 seconds of smelling;
B3. Channel 12 shows a statistically significant decrease of Total Hb after 30 seconds of smelling;
B4. Channel 5 shows a statistically significant increase of Oxy Hb after 0-5 seconds of smelling;
B5. Channel 4 shows a statistically significant increase of Total Hb after 0-5 seconds of smelling;
B6. Channel 5 shows a statistically significant increase of Total Hb after 0-5 seconds of smelling;
B7. Channel 11 shows a statistically significant increase of Oxy Hb after 0-10 seconds of smelling;
B8. Channel 8 shows a statistically significant decrease of Deoxy Hb after 5-10 seconds of smelling;
B9. Channel 10 shows a statistically significant decrease of Deoxy Hb after 5-10 seconds of smelling;
B10. Channel 18 shows a statistically significant increase of Oxy Hb after 5-10 seconds of smelling;
C1. Channel 4 shows a statistically significant increase of Oxy Hb after 30 seconds of smelling;
C2. Channel 4 shows a statistically significant increase of Total Hb after 30 seconds of smelling;
C3. Channel 19 shows a statistically significant increase of Total Hb after 30 seconds of smelling;
C4. Channel 2 shows a statistically significant decrease of Deoxy Hb after 0-5 seconds of smelling;
C5. Channel 8 shows a statistically significant decrease of Deoxy Hb after 0-5 seconds of smelling;
C6. Channel 6 shows a statistically significant increase of Oxy Hb after 0-5 seconds of smelling;
C7. Channel 12 shows a statistically significant decrease of Oxy Hb after 0-5 seconds of smelling;
C8. Channel 13 shows a statistically significant increase of Oxy Hb after 0-5 seconds of smelling;
C9. Channel 16 shows a statistically significant increase of Oxy Hb after 0-5 seconds of smelling;
C10. Channel 6 shows a statistically significant increase of Oxy Hb after 5-10 seconds of smelling.

More preferably, at least twelve, and most preferably at least 15, out of the 20 conditions B1 through B10 and C1 through C10 are met.

Depending on how many of the conditions A1 through A6 are met, it is preferred that a greater number of conditions B1 through B10 and/or C1 through C10 are met.

Therefore, in an embodiment, further Criterion D is met.

Criterion D requires:
that at least 16, more preferably at least 17, and most preferably at least 18, out of the conditions B1 through B10 and C1 through C10 are met if zero or one out of the conditions A1 through A6 are met;
that at least 12, more preferably at least 13, and most preferably at least 14, out of the conditions B1 through B10 and C1 through C10 are met if two or three out of the conditions A1 through A6 are met; and
that at least 8, more preferably at least 9, and most preferably at least 10, out of the 20 conditions B1 through B10 and C1 through C10 are met if four, five or six out of the conditions A1 through A6 are met.

It was further found that, the more of the conditions A1 through A6 and/or B1 through B10 are met, the greater the improvement of the happiness state that is achieved.

Therefore, in an embodiment, at least one of Criterion E1 and Criterion E2 are met.

Criterion E1 requires that at least four, more preferably at least five, and most preferably all six out of the conditions A1 through A6 are met.

Criterion E2 requires that at least six, more preferably at least seven, and most preferably at least eight out of the ten conditions B1 through B10 are met.

Based on the above described method of assessment, it was possible to develop guidelines for creating fragrance compositions that have an effect of improving the happiness of a human subject.

Therefore, the present invention also provides a method of creating a fragrance composition having an effect of improving the happiness of a human subject, comprising the steps of:
(i) creating a test fragrance composition;
(ii) assessing the ability of the test fragrance composition to improve the happiness state of a human subject according to the method described above; and
(iii) adjusting, if necessary, the test fragrance composition by adding and/or removing at least one fragrance ingredient and/or increasing and/or reducing the concentration of at least one fragrance ingredient until the fragrance composition is found to improve the happiness state of the human subject.

Therefore, it is first assessed whether or not the test fragrance composition provides an effect of increasing happiness. Subsequently, if necessary, the composition is adjusted to create an improved fragrance composition.

Steps (ii) and (iii) may be repeated if necessary and/or desired.

Increasing the level of HMP and/or HMR and/or HMI fragrance ingredients increases the likelihood that the fragrance composition would have a suitable character to deliver the happiness benefit. Other ingredients reduce the likelihood that the benefit will be achieved, as their level in the fragrance composition is increased, e.g. RMP and/or IMPU fragrance ingredients.

Therefore, in one embodiment, at least one HMP fragrance material is added to the (test) fragrance composition in step (iii).

Alternatively or in addition, at least one HMR fragrance material is added to the (test) fragrance composition in step (iii).

Alternatively or in addition, at least one HMI fragrance material is added to the (test) fragrance composition in step (iii).

Alternatively or in addition, at least one RMP fragrance material may be removed from the (test) fragrance composition in step (iii).

Alternatively or in addition, at least one IMPU fragrance material may be removed from the (test) fragrance composition in step (iii).

Alternatively or in addition, the concentration of at least one HMP fragrance material may be increased in step (iii).

Alternatively or in addition, the concentration of at least one HMR fragrance material may be increased in step (iii).

Alternatively or in addition, the concentration of at least one HMI fragrance material may be increased in step (iii).

Alternatively or in addition, the concentration of at least one RMP fragrance material may be reduced in step (iii).

Alternatively or in addition, the concentration of at least one IMPU fragrance material may be reduced in step (iii).

It has been found that the following fragrance materials have a happy effect and are, thus, HMP fragrance ingredients: fruity-candied fruit ingredients (excluding damascone alpha), fruity-strawberry ingredients, fruity-raspberry ingredients, fruity-pineapple ingredients, grapefruit oil, 6,6-dimethoxy-2,5,5-trimethylhex-2-ene (methyl pamplemousse), hexenyl-3-salicylate, ylang ylang oil, ethyl 3-oxobutanoate (ethyl acetoacetate), 5-hexyloxolan-2-one (gamma decalactone), 5-octyloxolan-2-one (dodecalatone gamma), 2,2,5-trimethyl-5-pentylcyclopentan-1-one (veloutone), hexyl acetate, cassis base, 1-phenylethyl acetate (styrallyl acetate), (E)-4-methyldec-3-en-5-ol (undecavertol), 2-ethyl-3-hydroxypyran-4-one (ethyl maltol), 8-methyl-1,5-benzodioxepin-3-one (calone), 1-[(1R,2R,5S,7R)-2,6,6,8-tetramethyl-9-tricyclo[5.3.1.01,5]undec-8-enyl]ethanone (methyl cedryl ketone or vertofix coeur), and mixtures thereof.

It has been found that the following fragrance materials have a happy-relaxing effect and are, thus, HMR fragrance ingredients: of lemon oil, (E)-3,7-dimethylnona-1,6-dien-3-ol (ethyl linalool), benzyl acetate, 3-methyl-2-[(Z)-pent-2-enyl]cyclopent-2-en-1-one (jasmone-cis), 2-(2′-methylpropyl)-4-hydroxy-4-methyltetrahydropyran (Florosa), (E)-1-(2,6,6-trimethylcyclohex-2-en-1-yl)hepta-1,6-dien-3-one (cetone V), N-1-(2,6,6-trimethyl-1-cyclohex-2-enyl)pent-1-en-3-one (Isoraldeine), 3,7-dimethyloct-6-en-1-ol (citronellol), (E)-3,7-dimethylocta-2,6-dien-1-ol (geraniol), geranium oil, 4-(4-hydroxy-4-methylpentyl)cyclohex-3-enecarbaldehyde (Lyral, Cyclohexal), 5-heptyldihydro-furan-2(3H)-one (peach pure, undecalactone gamma), 1,4-dioxacycloheptadecane-5,17-dione (ethylene brassylate), mixtures of cyclohexadecanolide and cyclopentadecanone (Silvanone), (5E)-3-methylcyclopentadec-5-en-1-one (Muscenone), (E)-2-methoxy-4-(prop-1-en-1-yl)phenol (isoeugenol), and mixtures thereof.

It has been found that the following fragrance materials have a happy-invigorating effect and are, thus, HMI fragrance ingredients: citrus-orange ingredients, citrus-mandarin ingredients, 1-(2-tert-butylcyclohexyl)oxybutan-2-ol (amber core), (3aR,5aS,9aS,9bR)-3a,6,6,9a-tetramethyl-2,4,5,5a,7,8,9,9b-octahydro-1H-benzo[e][1]benzofuran (ambrofix or ambroxan), 1,5,5,9-tetra-methyl-13-oxatricyclo(8.3.0.0.(4.9)) tridecane (cetalox or fixambrene), 2,6-dimethyloct-7-en-2-ol (dihydromyrcenol), 3-(1,3-benzodioxol-5-yl)-2-methylpropanal (helional or tropional), (2-tert-butylcyclohexyl) acetate (agrumex), 1-(2,6,6-trimethyl-1-cyclohex-2-enyl)but-3-en-1-one (damascone alpha), 2,4-dimethyl-3-cyclohexene-1-carbaldehyde (cyclal C or tricyclal or ligustral), 4-allyl-2-methoxyphenol (eugenol), and mixtures thereof.

It has been found that the following fragrance materials have a relaxing effect and are, thus, RMP fragrance ingredients: 2-(4-methylcyclohex-3-en-1-yl)propan-2-ol (terpineol), hexyl 2-hydroxybenzoate (hexyl salicylate), jasmin oil, 7-hydroxy-3,7-dimethyloctanal (hydroxycitronellal), 3-methyl-5-phenylpentanol (Mefrosol), 2-(phenoxy)ethyl 2-methylpropanoate (phenoxyethyl isobutyrate), (12E)-1-oxacyclohexadec-12-en-2-one (Habanolide), 4-methoxybenzaldehyde (aubepine para cresol, anisic aldehyde), benzoin resinoids, 3-ethoxy-4-hydroxybenzaldehyde (ethyl vanillin), 4-hydroxy-3-methoxybenzaldehyde (vanillin), 2-ethyl-4(2′,2′,3′-trimethylcyclopent-3-enyl)but-enol (Bangalol or Radjanol), mixtures of 2-methyl-1-phenylpropan-2-yl butanoate and (phenoxy)ethyl 2-methylpropanoate (Prunella), and mixtures thereof.

It has been found that the following fragrance materials have an invigorating effect and are, thus, IMPU fragrance ingredients: aromatic-eucalyptus ingredients, aromatic-mint ingredients, aromatic-rosemary ingredients, citrus-lime ingredients, spicy-pepper ingredients, citrus-floral/lemon ingredients, lavandin oil, patchouli oil, clary sage oil, orange flower oil, guaiacwood oil, oakmoss oil, litsea cubeba oil, citral, benzyl 2-hydroxybenzoate (benzyl salicylate), 2-methyl-3-(4-(1-methylethyl)phenyl)propanal (cyclamen aldehyde), 3-(4-ethylphenyl)-2,2-dimethylpropanal (floralozone), prop-2-enyl 2-(3-methylbutoxy)acetate (allyl amyl glycolate), prop-2-enyl 2-cyclohexyloxyacetate (cyclogalbanate), 4-methyl-2-(2-methylprop-1-en-1-yl)tetrahydro-2H-pyran (rose oxide), 1-[(2Z,5Z,9Z)-2,6,10-trimethylcyclododeca-2,5,9-trien-1-yl]ethanone (trimofix O or cyclisone), methyl 2,4-dihydroxy-3,6-dimethylbenzoate (Evernyl or Everniate), 2,6-dimethylheptan-2-ol (dimetol), 3,7-dimethylocta-1,6-dien-3-yl acetate (linalyl acetate) fir balsam oil, pine needle base, and mixtures thereof.

Where trivial names are used to describe useful perfume ingredients herein, the skilled perfumer will understand that these are commonly used names in the art of perfumery. However, the skilled perfumer would also understand that these ingredients may also be known by other trivial synonyms, by CAS registry numbers, or by more formal nomenclature, such as IUPAC nomenclature. Furthermore, the skilled perfumer would be familiar with these other trivial synonyms, as well as with more formal nomenclature, or at the least, would be aware of standard reference works, such as The Good Scents Company website, which contains a comprehensive list of trivial names, registry numbers and more formal nomenclature for the perfume ingredients contained in the perfumers' palette.

Perfume compositions and individual perfume ingredients may be characterized by their odour attributes. Although perfume creation is part science and part artistry, and there is no absolute prescribed definition for odour attributes of perfume compositions and perfume ingredients, nevertheless trained perfumers, realizing that there will be margin for some subjectivity, will be able to assign perfume compositions and ingredients to a general odour descriptor and an odour family.

Odour families provide a general description of an odour space, and their number is usually limited. Hence, most of the ingredients used in perfumery and particularly useful in the context of the present invention may be described by a small set of odour families selected from the group consisting of “aldehydic”, “ambery”, “animalic”, “aromatic/herbal”, “citrus”, “earthy”, “floral”, “fruity”, “green”, “musky”, “roasted”, “spicy”, “sweet”, “watery”, and “woody”.

Odour descriptors provide a more accurate description of the odour of a perfume composition or ingredient within a family. They are more abundant and their number and diversity is often unlimited. Examples of odour descriptors include, but are not limited to, “aldehyde zest”, “almond”, “amber dry”, “ambergris”, “anis tarragon”, “apple”, “armoise”, “balsam”, “banana”, “blackcurrant”, “butter”, “candied fruit”, “caraway seed”, “cedar”, “cinnamon”, “citronella”, “clove”, “cocoa”, “coconut”, “coniferous”, “cooked sugar”, “copaiba”, “coriander leaf”, “cucumber”, “eucalyptus”, “fecal”, “floral-lemon”, “freesia”, “galbanum”, “grapefruit”, “grass”, “heliotrope”, “jasmine”, “lavender”, “leaf”, “leather”, “lemon”, “licorice-fenugreek”, “lily of the valley”, “lime”, “liquor”, “lychee”, “mandarin”, “mango”, “medicinal”, “melon”, “metallic”, “milk cream”, “mint”, “molasses”, “moss”, “mushroom”, “musk”, “musk tonkin”, “nut”, “orange”, “orange flower”, “orris”, “passionfruit”, “patchouli”, “peach”, “pear”, “pepper”, “pineapple”, “raspberry”, “rhubarb”, “rose”, “rosemary”, “sandalwood”, “sea water”, “solar”, “strawberry”, “terpenic”, “thyme”, “tonka”, “vanilla”, “vetiver”, “violet”, and “wax”.

This selection of odour families and odour descriptors allows the skilled perfumer to characterize the odour of all perfume ingredients contained in the perfumer's palette. Nevertheless, for the trained perfumer, reading the contents of this specification as a whole together with their common general knowledge, it would not present undue burden to modify part or all of this vocabulary around which there is subjectivity, and such modification would not impact the selection of perfume ingredients useful to positively impact the perception of happiness.

Specific examples of fruity-candied fruit ingredients, fruity-strawberry ingredients, fruity-raspberry ingredients, fruity-pineapple ingredients, citrus-orange ingredients, and citrus-mandarin ingredients, respectively, will be provided below.

Throughout this application, the term “oil” is meant to encompass fully natural essential oils and extracts, as well as oils derived from natural essential oils and extracts, and modified essential oils and extracts that may comprise additional ingredients; irrespective of the extraction method. The term “oil” is meant to further also encompass any reconstitution or mixture of ingredients that provides a similar odour impression to the corresponding essential oil.

As used throughout this application, the term “terpineol” refers to single isomers of terpineol (e.g. alpha terpineol), as well as to mixtures of two or more isomers of terpineol.

The present invention further provides fragrance compositions for improving the happiness state of a human subject.

The fragrance composition comprises at least 75%, preferably at least 85%, of fragrance ingredients drawn from the following groups:
a) at least about 5% by weight in total of at least three HMP fragrance ingredients;
b) optionally up to about 95% by weight in total of HMR, HMI, IMPU, RMP and/or GEN fragrance ingredients, provided the following conditions are met:

$$HMPs + HMRs \geq IMPUs \qquad ( b1 )$$

$$HMPs + HMIs \geq RMPs \qquad ( b2 )$$

$$HMRs + HMIs + HMPs + GENs \geq 65 \% ; \qquad ( b3 )$$

$$HMPs / ( HMPs + RMPs + IMPUs ) \geq 0.35 \qquad ( b4 )$$

$$HMPs / ( HMPs + RMPs + IMPUs ) + ( 100 - TOTAL ) \geq 0.2 \qquad ( b5 )$$

In the above formulation guidelines, all percentages are based on total weight of the fragrance ingredients constituting the fragrance composition. This means that solvents, diluents and other odourless vehicles are not taken into account in the calculation.

HMPs indicates the sum of percentages of HMP fragrance ingredients; HMRs indicates the sum of percentages of HMR fragrance ingredients; HMIs indicates the sum of percentages of HMI fragrance ingredients; IMPUs indicates the sum of percentages of IMPU fragrance ingredients; RMPs indicates the sum of percentages of RMP fragrance ingredients; GENs indicates the sum of percentages of GEN fragrance ingredients; and TOTAL indicates the sum of HMPs, HMRs, HMIs, IMPU, RMPs, and GENs; provided that low odour or no odour solvents, diluents and other vehicles are excluded from the calculation of these sums.

The symbol≥ indicates at least equal to.

The present invention is based on extensive testing of fragrance materials, by consumer testing and measurement of brain activity using fNIRS. Statistical analysis of the resulting data has allowed classifying the fragrance materials into different categories:
HMP comprises ingredients or bases strongly associated with happy moods;
HMR comprises ingredients that may support both happy and relaxing moods;
HMI comprises ingredients that may support both happy and invigorating moods;
RMP comprises ingredients that strongly support relaxing moods and/or negatively impact happy and invigorating moods;
IMPU comprises ingredients that strongly support invigorating moods; and
GEN comprises ingredients that may support a variety of moods.

It must be emphasized that these designations are relevant to ingredients as used by one skilled in the art (e.g. a perfumer) under the dosage and pattern constraints disclosed here.

The HMP fragrance ingredients are selected from the group consisting of fruity-candied fruit ingredients (excluding damascone alpha), fruity-strawberry ingredients, fruity-raspberry ingredients, fruity-pineapple ingredients, grapefruit oil, 6,6-dimethoxy-2,5,5-trimethylhex-2-ene (methyl pamplemousse), hexenyl-3-salicylate, ylang ylang oil, ethyl 3-oxobutanoate (ethyl acetoacetate), 5-hexyloxolan-2-one (gamma decalactone), 5-octyloxolan-2-one (dodecalatone gamma), 2,2,5-trimethyl-5-pentylcyclopentan-1-one (veloutone), hexyl acetate, cassis base, 1-phenylethyl acetate (styrallyl acetate), (E)-4-methyldec-3-en-5-ol (undecavertol), 2-ethyl-3-hydroxypyran-4-one (ethyl maltol), 8-methyl-1,5-benzodioxepin-3-one (calone), 1-[(1R,2R,5S,7R)-2,6,6,8-tetramethyl-9-tricyclo[5.3.1.01,5]undec-8-enyl]ethanone (methyl cedryl ketone or vertofix coeur), and mixtures thereof.

Fruity-candied fruit ingredients (excluding damascone alpha) include, but are not limited to, e.g. (E)-1-(2,6,6-trimethylcyclohexa-1,3-dien-1-yl)but-2-en-1-one (damascenone), dimethyl benzyl carbinyl butyrate, (E)-1-(2,6,6-trimethyl-1-cyclohexenyl)but-2-en-1-one (damascone beta), (E)-1-(2,2-dimethyl-6-methylidenecyclohexyl)but-2-en-1-one (damascone gamma), (E)-1-(2,6,6-trimethyl-1-cyclohex-3-enyl)but-2-en-1-one (damascone delta), tagetes oil, ethyl 2-ethyl-6,6-dimethylcyclohex-2-ene-1-carboxylate (givescone), ethyl 2,6,6-trimethylcyclohexa-1,3-diene-1-carboxylate (ethyl safranate), ethyl 2,6,6-trimethylcyclohexa-1,3-diene-1-carboxylate (cristalon), (2E,5Z)-5,6,7-trimethylocta-2,5-dien-4-one (pomerose), and (3α,4β,7β,7α)-octahydro-4,7-methano-3aH-indene-3a-carboxylic acid ethyl ester (fruitate).

Fruity-strawberry ingredients include, but are not limited to, e.g. benzyl cinnamate, ethyl 3-methyl-3-phenyloxirane-2-carboxylate (strawberry pure), ethyl butanoate (ethyl butyrate), ethyl 2-methylpropionate (ethyl isobutyrate), ethyl cyclohexanecarboxylate (esterly), ethyl cinnamate, methyl cinnamate, benzyl cinnamate, ethyl phenyl glycidate, phenyl ethyl butyrate, benzyl butyrate, ethyl isovalerate, phenyl ethyl isovalerate, and 2-octene-4-one.

Fruity-raspberry ingredients include, but are not limited to, e.g. 4-(4-hydroxyphenyl)butan-2-one (raspberry ketone), methyoxy phenyl butanone, and ethyl 6-acetyloxyhexanoate (berryflor).

Fruity-pineapple ingredients include, but are not limited to, e.g. prop-2-enyl 3-cyclohexylpropanoate (allyl cyclohexyl propionate), prop-2-enyl heptanoate (allyl oenanthate), ethyl octanoate (ethyl oenanthate), 3-methylbutyl octanoate (isoamyl caproate), methyl hexanoate, ethyl hexanoate, pentyl hexanoate (amyl caproate), phenyl ethyl isobuyrate, allyl propionate, and methyl octanoate.

Cassis base is a reconstitution of, i.e. a mixture of fragrance ingredients resembling the smell of, cassis.

The HMR fragrance ingredients are selected from the group consisting of lemon oil, (E)-3,7-dimethylnona-1,6-dien-3-ol (ethyl linalool), benzyl acetate, 3-methyl-2-[(Z)-pent-2-enyl]cyclopent-2-en-1-one (jasmone-cis), 2-(2′-methylpropyl)-4-hydroxy-4-methyltetrahydropyran (Florosa), (E)-1-(2,6,6-trimethylcyclohex-2-en-1-yl)hepta-1,6-dien-3-one (cetone V), N-1-(2,6,6-trimethyl-1-cyclohex-2-enyl)pent-1-en-3-one (Isoraldeine), 3,7-dimethyloct-6-en-1-ol (citronellol), (E)-3,7-dimethylocta-2,6-dien-1-ol (geraniol), geranium oil, 4-(4-hydroxy-4-methylpentyl)cyclohex-3-enecarbaldehyde (Lyral, Cyclohexal), 5-heptyldihydrofuran-2(3H)-one (peach pure, undecalactone gamma), 1,4-dioxacycloheptadecane-5, 17-dione (ethylene brassylate), mixtures of cyclohexadecanolide and cyclopentadecanone (Silvanone), (5E)-3-methylcyclopentadec-5-en-1-one (Muscenone), (E)-2-methoxy-4-(prop-1-en-1-yl)phenol (isoeugenol), and mixtures thereof.

The HMI fragrance ingredients are selected from the group consisting of citrus-orange ingredients, citrus-mandarin ingredients, 1-(2-tert-butylcyclohexyl)oxybutan-2-ol (amber core), (3aR,5aS,9aS, 9bR)-3a,6,6,9a-tetramethyl-2,4,5,5a,7,8,9,9b-octahydro-1H-benzo[e][1]benzofuran (ambrofix or ambroxan), 5,5,9-tetramethyl-13-oxatricyclo(8.3.0.0.(4.9))tridecane (cetalox or fixambrene), 2,6-dimethyloct-7-en-2-ol (dihydromyrcenol), 3-(1,3-benzodioxol-5-yl)-2-methylpropanal (helional or tropional), (2-tert-butylcyclohexyl) acetate (agrumex), 1-(2,6,6-trimethyl-1-cyclohex-2-enyl)but-3-en-1-one (damascone alpha), 2,4-dimethyl-3-cyclohexene-1-carbaldehyde (cyclal C or tricyclal or ligustral), 4-allyl-2-methoxyphenol (eugenol), and mixtures thereof.

Citrus-orange ingredients include, but are not limited to, e.g. orange oil, orange terpenes, and orange aldehyde.

Citrus-mandarin ingredients include, but are not limited to, e.g. mandarin oil, tangerine oil, (E)-6, 10-dimethylundeca-5,9-dien-2-yl acetate (tangerinol), methyl 2-methylaminobenzoate (dimethyl anthranilate), and octanol-3.

The IMPU fragrance ingredients are selected from the group consisting of aromatic-eucalyptus ingredients, aromatic-mint ingredients, aromatic-rosemary ingredients, citrus-lime ingredients, spicy-pepper ingredients, citrus-floral/lemon ingredients, lavandin oil, patchouli oil, clary sage oil, orange flower oil, guaiacwood oil, oakmoss oil, litsea cubeba oil, citral, benzyl 2-hydroxybenzoate (benzyl salicylate), 2-methyl-3-(4-(1-methylethyl)phenyl)propanal (cyclamen aldehyde), 3-(4-ethylphenyl)-2,2-dimethylpropanal (floralozone), prop-2-enyl 2-(3-methylbutoxy)acetate (allyl amyl glycolate), prop-2-enyl 2-cyclohexyloxyacetate (cyclogalbanate), 4-methyl-2-(2-methylprop-1-en-1-yl)tetrahydro-2H-pyran (rose oxide), 1-[(2Z,5Z,9Z)-2,6,10-trimethylcyclododeca-2,5,9-trien-1-yl]ethanone (trimofix O or cyclisone), methyl 2,4-dihydroxy-3,6-dimethylbenzoate (Evernyl or Everniate), 2,6-dimethylheptan-2-ol (dimetol), 3,7-dimethylocta-1,6-dien-3-yl acetate (linalyl acetate) fir balsam oil, pine needle base, and mixtures thereof.

Aromatic-eucalyptus ingredients include, but are not limited to, e.g. eucalyptus oil, and 1-8 cineol (eucalyptol).

Aromatic-mint ingredients include, but are not limited to, e.g. peppermint oil, spearmint oil, L- and D/L-2-isopropyl-5-methylcyclohexanol (L- and DL-menthol), [(1R,2S,5R)-5-methyl-2-propan-2-ylcyclohexyl] acetate (menthyl acetate), 2-butan-2-ylcyclohexan-1-one (freskomenthe), 5-methyl-2-prop-1-en-2-ylcyclohexan-1-ol (isopulegol), D/L- and L-2-isopropyl-5-methylcyclohexanone (D/L- and L-menthone), and L- and D/L-2-isopropyl-5-methylcyclohexanone (L- and racemic isomenthone).

Aromatic-rosemary ingredients include, but are not limited to, e.g. rosemary oil, (1R,2S,4R)-1,7,7-trimethylbicyclo[2.2.1]heptan-2-ol (borneol), (1R,4S,6R)-1,7,7-trimethylbicyclo[2.2.1]heptan-6-ol (iso-borneol), 1,7,7-trimethylbicyclo[2.2.1]heptan-2-one (camphor), 8,8-dimethyl-9-propan-2-yl-6,10-dioxaspiro[4.5]decane (opalal), 2-(5-methyl-5-vinyltetrahydro-2-furanyl)-2-propanol (linalool oxide), trans-methyl 1,4-dimethyl-cyclohexanecarboxylate (cyprisate), 2-ethenyl-2,6,6-trimethyloxane (limetol), and 2-butyl-4,4,6-trimethyl-1,3-dioxane (herboxane).

Citrus-lime ingredients include, but are not limited to, e.g. lime oil, lime terpenes, lime oxide, 1-methyl-4-(prop-1-en-2-yl)cyclohex-1-ene (dipentene), 1-methyl-4-propan-2-ylidenecyclohexene (terpinolene), (E)-3,7-dimethylocta-1,3,6-triene (ocimene), 1,1-diethoxy-3,7-dimethylocta-2,6-diene (citrathal), elemi oil, (4-methyl-1-isopropylbenzene (para-cymene), and 3,7,7-trimethyl-bicyclo[4.1.0]hept-3-ene (delta-3-carene).

Spicy-pepper ingredients include, but are not limited to, e.g. ginger oil, nutmeg oil, olibanum oil, cardamom oil, copaiba balsam oil, curcuma oil, pepper oil, and (4Z)-4,11,11-trimethyl-8-methylenebicyclo[7.2.0]undec-4-ene (caryophyllene).

Citrus-floral/lemon ingredients include, but are not limited to, e.g. bergamot oil, coriander oil, and coriander seed oil.

Pine needle base is a reconstitution of, i.e. a mixture of fragrance ingredients resembling the smell of, pine needle.

The RMP fragrance ingredients are selected from the group consisting of 2-(4-methylcyclohex-3-en-1-yl)propan-2-ol (terpineol), hexyl 2-hydroxybenzoate (hexyl salicylate), jasmin oil, 7-hydroxy-3,7-dimethyloctanal (hydroxycitronellal), 3-methyl-5-phenylpentanol (Mefrosol), 2-(phenoxy)ethyl 2-methylpropanoate (phenoxyethyl isobutyrate), (12E)-1-oxacyclohexadec-12-en-2-one (Habanolide), 4-methoxybenzaldehyde (aubepine para cresol, anisic aldehyde), benzoin resinoids, 3-ethoxy-4-hydroxybenzaldehyde (ethyl vanillin), 4-hydroxy-3-methoxybenzaldehyde (vanillin), 2-ethyl-4(2′,2′,3′-trimethylcyclopent-3-enyl)but-enol (Bangalol or Radjanol), mixtures of 2-methyl-1-phenylpropan-2-yl butanoate and (phenoxy)ethyl 2-methylpropanoate (Prunella), and mixtures thereof.

The GEN fragrance ingredients are selected from the group consisting of 3,7-dimethylocta-1,6-dien-3-ol (linalool), methyl 3-oxo-2-pentylcyclopentaneacetate (methyl dihydro-jasmonate, cepionate, hedione), hexyl cinnamic aldehyde, 3-(4-(1,1-dimethylethyl)phenyl-2-methylpropanal (Lilial), (E)-4-(2,6,6-trimethylcyclohex-1-en-1-yl)but-3-en-2-one (ionone beta), 2-phenylethyl alcohol, 1-(2,3,8,8-tetramethyl-1,3,4,5,6,7-hexahydronaphthalen-2-yl)ethanone (sylvamber or iso e super or iso gamma super), 1,15-pentadecanolide (Thibetolide), (isocamphyl-5)cyclohexanol (sandela), (3R,3aS,6R,7R,8aS)-octahydro-6-methoxy-3,6,8,8-tetramethyl-1H-3a,7-methanoazulene (cedryl methyl ether), and mixtures thereof.

The fragrance composition of the invention may further comprise up to 25% of other fragrance ingredients, which are not specified herein as being members of any of the above groups, excluding odourless or low-odour solvents or diluents, as noted above. They may be single ingredients or mixtures, both synthetic and natural (for example essential oils), and are well described e.g. in: “Common Fragrance and Flavor Materials” by Bauer, Garbe and Surburg, VCH Publ., 2^{nd} edition (1990), and “Perfume and Flavour Materials”, Steffen Arctander, published in two volumes by the author (1969), also by Arctander “Perfume and Flavor Materials of Natural Origin” (1960), and Perfume & Flavor Chemicals”, S. Arctander (Allured Publishing, 1994), as well as later editions of this work, which perfume ingredients contained therein are herein incorporated by reference.

Perfume compositions of the present invention may further contain substantially odourless ingredients. In the context of the present invention, “substantially odourless” means that the ingredient has no odour or that its odour is weak and often barely perceptible. These substantially odourless ingredients include excipients conventionally used in conjunction with perfume ingredients in perfume compositions, for example carrier materials, and other auxiliary agents commonly used in the art, e.g. solvents, such as dipropylene glycol (DPG), isopropyl myristate (IPM), benzyl benzoate (BB), propylene glycol (PG) and triethyl citrate (TEC); mineral oils and vegetable oils; and antioxidants. As such, these substantially odourless ingredients are not considered to be perfume ingredients in the context of the present invention. In particular, solvents are not taken into account when calculating the weight percentages.

The perfume compositions of the present invention may be presented in the form of free-oil, or they may be encapsulated. Several encapsulating media are known in the art for encapsulating perfume compositions. Particular encapsulating media include microcapsules formed of aminoplast resins, such as melamine-formaldehyde resins, polyurea, polyamide, as well as copolymers of acrylic acid, methacrylic acid and their esters. Alternatively, the encapsulating media may be formed of natural or modified natural polymers, such as polysaccharides or proteins.

The above definition of the fragrance compositions of the present invention provides sufficient freedom in formulation to permit consideration of the hedonic properties of the composition. The invention can thus enable formulation of fragrance compositions that make people happy and also have good hedonic properties.

The present invention describes how to formulate reliably fragrance compositions which are likely to induce or be associated with positive, happy moods and emotions. The effects are sufficiently pronounced that they can be measured reliably and reproducibly. The fragrance compositions made according to the teachings disclosed herein can be hedonically pleasant, suitable for a wide range of consumer products, and of sufficient pleasantness/acceptability that they would be appropriate even if they did not possess added functionality. In addition, fragrance compositions of the invention can be resilient to variation in the target consumer group (e.g. British vs. American), and have been found to be perceived as consistently happy, joyful, playful, etc. for consumers in the UK, France, USA and Brazil, for example.

Fragrance compositions in accordance with the invention have been found:
a) to promote positive mood states such as happy, uplifted, enthusiastic, ecstatic, light-hearted, carefree, euphoric, excited, delighted, playful, optimistic and fulfilled: Test subjects have reported that they feel happier after smelling or using consumer products incorporating the fragrance compositions, and that the products themselves convey a happier smell;
b) not to promote negative mood states, such as depressing, stressful, annoying, or bored mood states.

Increasing the level of “happy” ingredients, in particular those in group HMP, increases the likelihood that the fragrance compositions would have a suitable character to enhance the state of happiness. Other ingredients reduce the likelihood of this benefit being achieved, in particular ingredients that highly contribute to invigorating (IMPU) or relaxing (RMP) moods.

In an embodiment, the fragrance composition comprises at least about 10%, more preferably at least about 15%, by weight in total of HMP fragrance ingredients.

In an embodiment, the fragrance composition comprises at least four HMP fragrance ingredients, more preferably at least five HMP fragrance ingredients. By increasing the number of fragrance ingredients, the hedonics of the fragrance composition are improved.

In an embodiment, the fragrance composition comprises at least one HMP, HMI and/or HMR fragrance ingredient selected from one or more of the following groups:
one or more fruity-candied fruit ingredients selected from the group consisting of (E)-1-(2,6,6-trimethylcyclohexa-1,3-dien-1-yl)but-2-en-1-one (damascenone), dimethyl benzyl carbinyl butyrate, (E)-1-(2,6,6-trimethyl-1-cyclohexenyl)but-2-en-1-one (damascone beta), (E)-1-(2,2-dimethyl-6-methylidenecyclohexyl)but-2-en-1-one (damascone gamma), (E)-1-(2,6,6-trimethyl-1-cyclohex-3-enyl)but-2-en-1-one (damascone delta), tagetes oil, ethyl 2-ethyl-6,6-dimethylcyclohex-2-ene-1-carboxylate (givescone), ethyl 2,6,6-trimethylcyclohexa-1,3-diene-1-carboxylate (ethyl safranate), ethyl 2,6,6-trimethylcyclohexa-1,3-diene-1-carboxylate (cristalon), (2E,5Z)-5,6,7-trimethylocta-2,5-dien-4-one (pomerose), (3α,4β,7β,7α)-octahydro-4,7-methano-3aH-indene-3a-carboxylic acid ethyl ester (fruitate), and mixtures thereof;
one or more fruity-strawberry ingredients selected from the group consisting of benzyl cinnamate, ethyl 3-methyl-3-phenyloxirane-2-carboxylate (strawberry pure), ethyl butanoate (ethyl butyrate), ethyl 2-methylpropionate (ethyl isobutyrate), ethyl cyclohexanecarboxylate (esterly), ethyl cinnamate, methyl cinnamate, benzyl cinnamate, ethyl phenyl glycidate, phenyl ethyl butyrate, benzyl butyrate, ethyl isovalerate, phenyl ethyl isovalerate, 2-octene-4-one, and mixtures thereof;
one or more fruity-raspberry ingredients selected from the group consisting of 4-(4-hydroxyphenyl)butan-2-one (raspberry ketone), methyoxy phenyl butanone, ethyl 6-acetyloxyhexanoate (berryflor), and mixtures thereof;
one or more fruity-pineapple ingredients selected from the group consisting of prop-2-enyl 3-cyclohexylpropanoate (allyl cyclohexyl propionate), prop-2-enyl heptanoate (allyl oenanthate), ethyl octanoate (ethyl oenanthate), 3-methylbutyl octanoate (isoamyl caproate), methyl hexanoate, ethyl hexanoate, pentyl hexanoate (amyl caproate), phenyl ethyl isobuyrate, allyl propionate, methyl octanoate, and mixtures thereof;
one or more citrus-orange ingredients selected from the group consisting of orange oil, orange terpenes, orange aldehyde, and mixtures thereof; and/or
one or more citrus-mandarin ingredients selected from the group consisting of mandarin oil, tangerine oil, (E)-6,10-dimethylundeca-5,9-dien-2-yl acetate (tangerinol), methyl 2-methylaminobenzoate (dimethyl anthranilate), octanol-3, and mixtures thereof.

The above groups of happy fragrance ingredients may be combined as desired.

In an embodiment, the amounts of the ingredients are selected such that HMRs+HMIs+HMPs+GENs≥70%, more preferably HMRs+HMIs+HMPs+GENs≥75%, and most preferably HMRs+HMIs+HMPs+GENs≥80%.

In an embodiment, the amounts of the ingredients are selected such that HMPs/(HMPs+RMPs+IMPUs)≥0.40, more preferably HMPs/(HMPs+RMPs+IMPUs)≥0.45, and most preferably HMPs/(HMPs+RMPs+IMPUs)≥0.50.

Another aspect of the invention relates to a method of delivering positive mood benefits, particularly happiness, to human subjects, comprising delivering the fragrance composition to said human subjects. For instance, the fragrance may be delivered in a consumer product.

Therefore, the present invention also provides a consumer product comprising the fragrance composition of the invention.

The perfume compositions of the present invention may be used to impart desirable odour impressions on all manner of consumer products, such as for instance hydro-alcoholic perfumes, deodorants, antiperspirants, skin care products, hair care products, laundry care products, home care products or air fresheners.

More particularly, the perfume compositions of the present invention may be employed in laundry care applications, personal care products for treating the hair and/or skin of human subjects, oral care products, and air care products.

Consumer products comprise formulated mixtures of various functional ingredients, such as surfactants, emulsifiers, polymers, fillers and solvents. These formulated mixtures are usually referred to as “bases”.

Particular consumer products include, but are not limited to consumer products intended for application to the body (i.e. skin or hair), to hard surfaces (e.g. kitchen and bathroom worktops, ceramic surfaces), to fabrics, and for air care benefits (e.g. air-fresheners). Such products can take a variety of forms, including, but not limited, to powders, bars, sticks, tablets, creams, mousses, gels, liquids, sprays and sheets. The proportion of perfume composition contained in such products may lie in a range from 0.05% (as for example in a low odour skin cream) to 100 wt.-% (as for example in an air freshener) based on the total weight of the consumer product. The means of incorporating a perfume composition into a consumer product is known. Existing techniques may be used for incorporating the perfume composition directly into a product, or the perfume composition may be absorbed on a carrier material and then admixed to the product.

In an embodiment of the present invention, the consumer product is a laundry care product. Laundry care products include powder and liquid detergents and fabric softeners, stain removers and pre-wash treatments, conditioners and softeners (including standard and concentrated conditioners, softeners and dryer sheets), laundry aids (including stain removers, ironing aids, whiteners and colour care products and other ancillary fabric care products), laundry detergents (including machine wash liquid detergents, other machine wash detergents—including powders, capsules and tablets—and hand wash detergents—powders, flakes and cakes/bars), sheet sprays, clothing sprays, laundry perfumes, dryer sachets, perfumed sachets, dryer sheets, laundry soap, laundry detergents, detergent for delicate textiles, ironing sprays, starch, perfume sheets, pillow mists, drawer liner sheets, cedar closet sprays, linen waters, and refills and combinations thereof.

In an embodiment of the invention, the consumer product is a personal care product. Personal care products include soaps, shower gels, body creams, body lotions, body mists, perfumery, cosmetics, floating bath oils, after shaves, creams, lotions, deodorants (including stick deodorants), pre-electric shave lotions, after-shave lotions, antiperspirants, shampoos, conditioners, rinses, skin care products, eye makeups, body shampoos, protective skin formulations, lipsticks, lip glosses, after-bath splashes, pre-sun and sun products (including sunscreens). Virtually any chemical product which comes into contact with the hair or skin and which may include effective amounts, concentrations or proportions of one or more of the perfume compositions of the present invention may be considered a personal care product according to the present invention.

In an embodiment of the present invention, the consumer product is an air care product. Air care products include candles and air-freshener devices, such as liquid electrical air-freshener devices, aerosol sprays, pump action sprays, perfumed candles, membrane permeation devices, liquid wick devices, oil based gel perfumes, and aqueous gels.

In an embodiment of the present invention, the consumer product is a home care product. Home care products can be used particularly for cleaning, rinsing, care or treatment of industrial, domestic or communal hard surfaces, as well as textile article surfaces; they are targeted at conferring on the surfaces treated therewith benefits such as water repellence, soil release, stain resistance, anti-fogging, surface repair, anti-wrinkling, shine, lubrication and/or at improving the residuality, impact and/or efficacy of active materials comprised in said home care product. Hence, home care compositions according to the invention include surface cleaning compositions (for example glass, floor, counter, bath, toilet bowl, sink, appliance and furniture cleaning compositions), disinfectants (for example spray and solid air disinfectants, including gels, and spray, solid, liquid and paste surface disinfectants), waxes and other surface protecting and/or polishing compositions, and rug shampoos.

Also included within the scope of the invention is a method of delivering positive mood benefits or happiness benefits to a subject, particularly a human, comprising administering to the subject an effective amount of a fragrance composition in accordance with the invention. The composition should be administered in an appropriate amount to produce a benefit (i.e. a suprathreshold amount) without causing irritation (i.e. a non-irritant amount). An appropriate effective amount of any given composition can be readily determined, e.g. by experiment. To be effective, the compositions should be administered for inhalation by the subject.

Therefore, the present invention also provides a method of improving the happiness state of a human subject, comprising the step of providing an effective amount of the fragrance composition of the invention to the human subject.

In the context of the studies resulting in the present invention, several fragrance ingredients have been identified that are able to improve the happiness state of a human subject.

Therefore, the present invention also relates to the use of a fragrance ingredient for improving the happiness state of a human subject, wherein the fragrance ingredient is selected from the group consisting of:
one or more fruity-candied fruit ingredients selected from the group consisting of dimethyl benzyl carbinyl butyrate, (E)-1-(2,6,6-trimethyl-1-cyclohexenyl)but-2-en-1-one (damascone beta), (E)-1-(2,2-dimethyl-6-methylidenecyclohexyl)but-2-en-1-one (damascone gamma), (E)-1-(2,6,6-trimethyl-1-cyclohex-3-enyl)but-2-en-1-one (damascone delta), tagetes oil, ethyl 2-ethyl-6,6-dimethylcyclohex-2-ene-1-carboxylate (givescone), ethyl 2,6,6-trimethylcyclohexa-1,3-diene-1-carboxylate (ethyl safranate), ethyl 2,6,6-trimethylcyclohexa-1,3-diene-1-carboxylate (cristalon), (2E,5Z)-5,6,7-trimethylocta-2,5-dien-4-one (pomerose), (3α,4β,7β,7α)-octahydro-4,7-methano-3aH-indene-3a-carboxylic acid ethyl ester (fruitate), and mixtures thereof;
one or more fruity-strawberry ingredients selected from the group consisting of benzyl cinnamate, ethyl butanoate (ethyl butyrate), ethyl 2-methylpropionate (ethyl isobutyrate), ethyl cyclohexanecarboxylate (esterly), ethyl cinnamate, methyl cinnamate, benzyl cinnamate, ethyl phenyl glycidate, phenyl ethyl butyrate, benzyl butyrate, ethyl isovalerate, phenyl ethyl isovalerate, 2-octene-4-one, and mixtures thereof;
one or more fruity-raspberry ingredients selected from the group consisting methyoxy phenyl butanone, ethyl 6-acetyloxyhexanoate (berryflor), and mixtures thereof;
one or more fruity-pineapple ingredients selected from the group consisting of ethyl octanoate (ethyl oenanthate), 3-methylbutyl octanoate (isoamyl caproate), methyl hexanoate, ethyl hexanoate, pentyl hexanoate (amyl caproate), phenyl ethyl isobuyrate, allyl propionate, methyl octanoate, and mixtures thereof;
one or more citrus-orange ingredients selected from the group consisting of orange aldehyde, and mixtures thereof; and/or
one or more citrus-mandarin ingredients selected from the group consisting of tangerine oil, (E)-6,10-dimethylundeca-5,9-dien-2-yl acetate (tangerinol), methyl 2-methylaminobenzoate (dimethyl anthranilate), octanol-3, and mixtures thereof.

The present invention is further illustrated by means of the following non-limiting examples:

### EXAMPLE 1: FUNCTIONAL NEAR INFRARED SPECTROSCOPY TESTING

### Task

The experimental protocol was divided in two parts:

In the first part, participants smelled a series of fifteen (three consecutive repetitions of five different odour conditions) fragrance samples provided on sorbarods, while their brain activity was being monitored through a fNIRS cap placed on the forehead. fNIRS channels were arranged as shown in FIG. 1.

In the second part, they rated each fragrance for the dimensions of pleasantness, invigorating power, relaxing power, happy power, and strength of the odour using a questionnaire. During this second part, their brain activity was not monitored.

The samples were typically prepared as follows: In each sorbarod, 0.8 g of neat fragrance oil was placed in the polyester insert by means of a pipette. Previous tests demonstrated that a range between 0.75 g and 0.85 g of neat oil will not significantly alter the perception of the fragrance in terms of properties and intensity, therefore the range is acceptable for any brain imaging test without influencing the results. Once the oil was dropped, the plastic cap was immediately placed on the sorbarod to prevent any diffusion of the fragrance in the environment. Sorbarods were then kept in standing (vertical) position for at least 24 h before being used for the test. After the sorbarod rests for 24 h, the full insert becomes soaked with the oil, guaranteeing that, if adequately stored (i.e. without exposing the sorbarod to direct sunlight or to extremely high temperatures, above 35° C.), the fragrance oil maintains the same olfactive properties (characteristics and intensity) for at least four weeks, up to eight weeks, depending on the oil. In this time frame, the sorbarods can be used for brain imaging tests without any significant alteration of the results. In the tests described in the current document, the samples were used within two weeks from the day they were created. Samples were normally stored in a refrigerator at 4° C. from the moment they were made to the morning of the test. The experimenter made sure that sorbarods were taken out of the fridge at least 2 h before the test to ensure they reached room temperature before being used. Removing them from the fridge the evening before the test, and leaving them overnight at room temperature, also does not have any significant effect on test results, as previous trials demonstrated.

In the first part, participants were asked to smell, keeping their eyes closed, the proposed number of sorbarods. They were not required to complete any other tasks, in order to eliminate any possible source of confound in the data not related to the perception of odours. In each test, one of the samples contained the fragranced benchmark and another one did not contain any fragrance (control sample). The other two or three samples contained the test fragrances. Thanks to the three repetitions, it was possible to confirm that the overall results were not affected by the number of fragrances tested in one trial, i.e. that the test of four or five different conditions in a single test was completely equivalent and that the studies were fully comparable.

The order of presentation for the sorbarods was semi-randomized: the order of the fragrances in each block was fully randomized, however participants had to complete smelling all samples in a block before moving to the following one, and the first sample of each block was always different from the last of the previous one, so to avoid smelling the same fragrance twice in two consecutive assessments.

After the participants had smelled all the sorbarods, the fNIRS cap was removed from the head and they completed the questionnaires at a self-timed pace, meaning that they were able to smell again each sample as many times as they wanted and take all the time they needed to answer each question. For this reason, no specific timeline for the second part of the experimental procedure will be reported in the following section.

### Timeline

All participants completed three blocks of five samples each. The three blocks were consecutive, and the participants were unaware that the sequence of four or five samples was repeated three times, as they were only told that the test involved smelling fifteen samples. Participants were asked, for each sample, to take the sorbarod in their hand, close their eyes, smell the sorbarod for thirty seconds and then, after returning the sorbarod to the experimenter, rest for thirty seconds with the eyes open. Longer intervals between two consecutive samples were taken if the participant explicitly asked for it, or if the fNIRS signals were not at a baseline level (necessary condition to start with a new trial). The latter case would happen in case of heavy movements from the participants, such as sneezing; however, the recovery time was in the order of a few seconds.

### Participants

For each study, at least fifteen healthy adults took part in this experiment. No specific selection criteria (i.e. handedness, age, etc.) have been applied in the choice of the participants, since no relevant exclusion criteria have been identified prior to testing.

### Statistical Analysis

Statistical significance was verified using a 2-tailed Student's t-test with a statistical significance threshold at 0.05.

### EXAMPLE 2: MOOD PORTRAITS® TESTING

### Task

Mood Portraits® is a self-report nonverbal method using pictures to measure consumers' moods and emotional responses to fragrances. This method allows participants to express what they feel in response to smelling a fragrance by selecting images that match their feelings rather than verbalising and rating their thoughts and emotions.

The experimental protocol was divided in two parts. In the first one, participants smelled a series of eight sorbarods and, while smelling each one, they selected a number of pictures chosen from a set of thirty pictures to describe the fragrance. The thirty pictures, printed in colour on A4 laminated sheets, were arranged on a display board. The number of pictures chosen by each participant to describe the fragrances was not pre-determined: each participant could choose as many as they wanted to describe each fragrance. The minimum number of pictures they had to select was one. In the second part of the test, they rated each fragrance for the dimensions of pleasantness, invigorating power, relaxing power, happy power, and strength of the odour using a questionnaire.

The order of presentation for the sorbarods was fully randomised and the pictures were arranged on four different boards to create a randomisation of the layout. For each series of eight fragrances, 80 healthy adults were asked to participate.

### Timeline

All participants smelled and rated eight fragrances during a single session. There was no time limit for the participants to smell the fragrance nor to select the pictures associated to each fragrance. This allowed the participants to provide truer responses without any time pressure associated.

Participants were allowed breaks at their leisure to prevent any fatigue or carry over effect, and moved to the following fragrance only when they considered themselves ready.

### Participants

For each test involving eight fragrances, eighty healthy adults were asked to participate in the study. Participants were screened for olfactive impairment, respiratory conditions or other personal conditions that could alter their sense of smell (e.g. pregnancy or consumption of tobacco-based products, like cigarettes). No other selection criteria (i.e. handedness, age, gender, etc.) have been applied in the choice of the participants, since no relevant exclusion criteria have been identified prior to testing.

### EXAMPLE 3: COMPOSITIONS TESTED

Compositions A through P were subjected to fNIRS and/or Mood Portraits® testing. Among these, Compositions A, B, C, D, M, N, O and P are comparative examples; all other compositions are fragrance compositions according to the present invention.

Ingredients contained in these compositions are specified in the two tables below.

| | | A | B | C | D |
|---|---|---|---|---|---|
| Ingredient | Group | wt % | wt % | wt % | wt % |
| CEDRYL METHYL ETHER | GEN | | 0.2000 | | 0.8850 |
| CEPIONATE | GEN | 8.0000 | | | |
| HEDIONE | GEN | 14.0000 | 5.0000 | | 2.6549 |
| HEXYL CINNAMIC | GEN | | | | 6.1947 |
| ALDEHYDE | | | | | |
| IONONE BETA | GEN | | 0.6000 | | |
| ISO E SUPER | GEN | 3.6000 | 8.0000 | 4.0000 | 4.4248 |
| LILIAL | GEN | 1.4000 | 0.2000 | | |
| LINALOOL | GEN | 6.0000 | 2.8000 | 14.0000 | 7.0796 |
| PHENYL ETHYL ALCOHOL | GEN | | | | |
| SANDELA | GEN | 0.2000 | | | |
| THIBETOLIDE | GEN | | | | |
| AGRUMEX | HMI | | | | |
| AMBROFIX | HMI | | 0.2000 | | 0.1416 |
| CETALOX | HMI | 2.2000 | | | |
| DAMASCONE ALPHA | HMI | 0.0500 | 0.0200 | | 0.0708 |
| DIHYDRO | HMI | 9.0000 | 1.5000 | 5.0000 | 5.9292 |
| MYRCENOL | | | | | |
| DIMETHYL | HMI | | | | |
| ANTHRANILATE | | | | | |
| EUGENOL | HMI | | | | 0.1327 |
| MANDARIN OIL | HMI | 4.0000 | 1.5000 | | 2.1726 |
| MANDARINAL BASE | HMI | | | | |
| ORANGE OIL | HMI | | | 1.5000 | |
| ORANGE TERPENES | HMI | | | | 3.9823 |
| TRICYCLAL | HMI | 0.0600 | 0.1000 | 0.1000 | 0.3540 |
| TROPIONAL | HMI | 1.0000 | 0.7000 | | 0.7080 |
| ALLYL CAPROATE | HMP | | | | |
| ALLYL CYCLOHEXYL | HMP | | | | |
| PROPIONATE | | | | | |
| ALLYL OENANTHATE | HMP | | | | |
| CALONE FINE | HMP | 0.3000 | | | 0.0708 |
| CASSIS BASE | HMP | | | | |
| DAMASCENONE | HMP | 0.0200 | 0.0150 | | 0.0442 |
| DAMASCONE DELTA | HMP | | | | |
| DECALACTONE | HMP | | | | |
| GAMMA | | | | | |
| DIMETHYL BENZYL | HMP | | | | |
| CARBINYL BUTYRATE | | | | | |
| ETHYL ACETOACETATE | HMP | | | | |
| ETHYL BUTYRATE | HMP | | | | |
| ETHYL ISOBUTYRATE | HMP | | | | |
| ETHYL MALTOL | HMP | | | | |
| GRAPEFRUIT OIL | HMP | 4.0000 | 0.3000 | | |
| HEXENYL-3-CIS | HMP | | | | 1.7699 |
| SALICYLATE | | | | | |
| HEXYL ACETATE | HMP | | | | |
| METHYL CEDRYL KETONE | HMP | 0.6000 | | | |
| METHYL PAMPLEMOUSSE | HMP | | | | |
| STYRALLYL ACETATE | HMP | | 0.1000 | | 0.8850 |
| UNDECAVERTOL | HMP | | | | |
| VERTOFIX COEUR | HMP | | | | 0.8850 |
| BENZYL ACETATE | HMR | | | | |
| CITRONELLOL | HMR | 0.2000 | | | 0.4425 |
| CYCLOHEXAL | HMR | | 0.1000 | | |
| ETHYL LINALOOL | HMR | 5.0000 | 2.0000 | | |
| ETHYLENE BRASSYLATE | HMR | 4.0000 | | 3.0000 | 3.5398 |
| FLOROSA | HMR | 0.1000 | | | |
| GERANIOL | HMR | | 0.3000 | 0.2500 | 0.4425 |
| GERANIUM OIL | HMR | 0.2000 | | | 0.4425 |
| ISOEUGENOL | HMR | | | | |
| ISORALDEINE 70 | HMR | 0.3000 | | | 0.8850 |
| JASMONE CIS | HMR | | | | |
| LEMON OIL | HMR | 1.0000 | 0.8000 | | |
| LEMON TERPENES | HMR | | | 5.0000 | |
| PEACH PURE | HMR | | | | |
| ALLYL AMYL GLYCOLATE | IMPU | | 0.0500 | | |
| BENZYL SALICYLATE | IMPU | 0.5000 | 8.0000 | 15.0000 | |
| BERGAMOT OIL | IMPU | 11.0000 | 6.0000 | 1.0000 | 6.1575 |
| BORNEOL CRYSTALS | IMPU | | | | |
| CAMPHOR | IMPU | | | 0.4000 | |
| CARDAMOM SEED OIL | IMPU | 0.0200 | 0.1500 | | 0.442 |
| CAROPHYLLENE | IMPU | | | 0.2500 | |
| CITRAL | IMPU | | | | 0.2655 |
| CYCLAMEN ALDEHYDE | IMPU | | | | |
| CYCLOGALBANATE | IMPU | 0.1000 | 0.0200 | | 0.7080 |
| DIMETOL | IMPU | | | | |
| EUCALYPTOL | IMPU | | | | |
| EUCALYPTUS GLOBULUS | IMPU | 0.2000 | | 4.0000 | |
| OIL | | | | | |
| EVERNYL | IMPU | 0.1000 | | | 0.1770 |
| FLORALOZONE | IMPU | | | | 0.5310 |
| FRESKOMENTHE | IMPU | | | 1.0000 | |
| LAVANDIN OIL | IMPU | 0.2000 | 0.2000 | 0.4000 | |
| LAVANDIN BASE | IMPU | | | 5.1000 | |
| LIME OIL | IMPU | 0.3000 | | | |
| LIME OXIDE | IMPU | | 0.0500 | | |
| LIME TERPENES BASE | IMPU | | | | |
| LINALYL ACETATE | IMPU | 2.5000 | | 20.0000 | 2.6549 |
| NUTMEG OIL | IMPU | | 1.5000 | | |
| OLIBANUM BASE | IMPU | | 0.0500 | | |
| PATCHOULI OIL | IMPU | | 0.1000 | | |
| PEPPERMINT OIL | IMPU | | | 0.1000 | 0.0354 |
| PEPPER OIL | IMPU | 0.2000 | 0.1000 | | |
| ROSE OXIDE | IMPU | | | | 0.0442 |
| BUTYL HYDROXY | INERT | 0.4000 | 0.2000 | | |
| TOLUENE | | | | | |
| DIETHYL PHTHALATE | INERT | 0.1800 | 0.2700 | | |
| DIPROPYLENE GLYCOL | INERT | 11.4450 | 49.2250 | | 38.8850 |
| DOWANOL TPM | INERT | | | | |
| TRIETHYL CITRATE | INERT | | | 19.0600 | |
| AUBEPINE PARA CRESOL | RMP | | | | |
| ETHYL VANILLIN | RMP | | | | |
| HABANOLIDE | RMP | 1.8000 | | | 3.0973 |
| HYDROXYCITRONELLAL | RMP | | | | |
| PHENOXY ETHYL | RMP | | | | |
| ISOBUTYRATE | | | | | |
| RADJANOL | RMP | 0.0200 | 0.8000 | | 0.4425 |
| TERPINEOL | RMP | | | | |
| VANILLIN | RMP | 0.1000 | 0.1000 | | |
| ADOXAL | | 0.0300 | | | |
| ALDEHYDE C10 DECYLIC | | 0.0080 | | | 0.0442 |
| ALDEHYDE C110 | | 0.0040 | | | |
| UNDECYLIC | | | | | |
| ALDEHYDE | | | | | |
| C11UNDECYLENIC | | | | | |
| ALDEHYDE C12 LAURIC | | 0.0060 | | | |
| ALDEHYDE C12 MNA | | 0.0080 | | | 0.0442 |
| ALDEHYDE C8 OCTYLIC | | | | | |
| ALDEHYDE C9 NONYLIC | | 0.0040 | | | |
| ALDEHYDE ISO C11 | | | | | |
| AMBERKETAL | | | 0.1000 | | |
| AMBERMAX | | | | | |
| AMBRE BASE | | 0.2000 | | | |
| AMBRETTOLIDE | | | 0.3000 | | |
| AMPHERMATE | | | 0.0500 | | |
| APPLE BASE | | | | | |
| AMYL BUTYRATE | | | | | |
| AMYL SALICYLATE | | 0.3000 | | | |
| BASIL OIL | | | | 0.0300 | 0.0885 |
| BENZALDEHYDE | | 0.0020 | | | |
| BOISAMBRENE FORTE | | 0.0200 | | | 1.3274 |
| BORNYL ACETATE | | | | 0.2500 | |
| BOURGEONAL | | | | | |
| BUTYL ACETATE | | | 0.0100 | | |
| BUTYL BUTYRO LACTATE | | | | | |
| BUTYL CYCLOHEXYL | | | | | |
| ACETATE PARA | | | | | |
| BUTYL QUINOLINE | | 0.0020 | | | |
| SECONDARY | | | | | |
| CALONE | | 0.5000 | | | 0.0442 |
| CARVONE LAEVO | | | | | 0.0885 |
| CASMERAN | | 0.1000 | 0.4000 | | |
| CEDARWOOD OIL | | 0.2000 | 1.2000 | | |
| CINNAMON BARK OIL | | | 0.0100 | | |
| CISTUS OIL | | 0.0040 | | | |
| CITRONELLYL ACETATE | | | | | |
| CITRONELLYL NITRILE | | | | | |
| CLOVE LEAF OIL | | | | | 0.0885 |
| CORPS CASSIS | | | | | |
| COSMONE | | | 0.2000 | | |
| COUMARINE | | | 0.4000 | | 0.0885 |
| CUMINIC ALDEHYDE | | | | | |
| DECALACTONE DELTA | | | | | |
| DIMETHYL BENZYL | | | | | |
| CARBINYL ACETATE | | | | | |
| DIPHENYL OXIDE | | | | | |
| DODECENAL | | | | | |
| EBANOL | | | | | |
| ESTRAGOLE | | | | | |
| ETHYL ACETATE | | | | | |
| ETHYL METHYL-2- | | | 0.0020 | | |
| BUTYRATE | | | | | |
| ETHYL OCTANOATE | | | | | |
| FENNALDEHYDE | | 0.1000 | | | |
| FLORHYDRAL | | | | | 0.2655 |
| FLOROCYCLENE | | | | | |
| FIXOLIDE | | | 1.0000 | | |
| FOLIONE | | | | | |
| FRESCILE | | 0.0020 | | | |
| GALAXOLIDE | | 3.0000 | 1.8000 | | |
| GALBANONE | | | | | |
| GALBANUM OIL | | | | 0.0300 | |
| GALBEX | | | 0.1000 | | |
| GARDOCYCLENE | | | | | |
| GERANYL ACETATE | | 0.1000 | | 0.5000 | |
| GUAVE BASE | | | | | |
| HELIOTROPINE | | 0.0060 | | | |
| HEXENAL-2-TRANS | | | | | |
| HEXENOL-3-CIS | | 0.0600 | 0.0300 | | 0.0885 |
| HEXENYL-3-CIS ACETATE | | 0.0800 | | | 0.1770 |
| HYDROXY ETHYL METHYL | | | | | |
| THIAZOL | | | | | |
| INDOLE | | | | | |
| IRALIA | | | 0.0500 | | |
| IRONE ALPHA | | | 0.0500 | | |
| ISOAMYL ACETATE | | | | | |
| ISOEUGENOL | | | | | |
| ISOPROPYL-2-METHYL-4 | | | | | |
| THIAZOLE | | | | | |
| ISOPROPYL METHYL-2- | | | 0.0100 | | |
| BUTYRATE | | | | | |
| ISORALDEINE CETONE | | | 1.0000 | | |
| ALPHA | | | | | |
| JASMACYCLENE | | | | | |
| JASMALACTONE | | | | | |
| JAVANOL | | 0.0100 | 0.1000 | | |
| KARANAL | | | | | 0.1770 |
| KEPHALIS | | | 0.9000 | | |
| LABIENOXIME | | | | | |
| LAVENDER OIL | | | | 0.0300 | |
| LEMONGRASS OIL | | 0.1000 | | | |
| LEMONILE | | | | | |
| LIFFAROME | | 0.0200 | 0.0900 | | 0.0442 |
| MANGO BASE | | | | | |
| MANZANATE | | | 0.0020 | | |
| MARENIL | | 0.0020 | | | 0.0708 |
| MELONAL | | | 0.0010 | | 0.0354 |
| METHYL ANTHRANILATE | | | | | |
| METHYL CAMOMILLE | | | | | |
| METHYL HEPTENONE | | | | | |
| METHYL-2 BUTANOL-1 | | | | | |
| MUSK KETONE | | | | | |
| MYRALDENE | | | | | |
| MYRCENE 90 | | | | | 0.0770 |
| NECTARYL | | | | | |
| NEOCASPIRENE | | | | | 0.0265 |
| NEROLI OIL | | 0.0200 | | | |
| NONALACTONE GAMMA | | | | | |
| NONENOL-6-CIS | | | | | |
| NYMPHEAL | | | | | |
| OAKMOSS BASE | | 0.0998 | | | |
| ORANGE FLOWER ETHER | | | | | |
| ORANGER CRYSTALS | | | | | 0.0442 |
| ORRIS BASE | | | 0.0400 | | |
| OSYROL | | 0.0300 | | | |
| OXANE | | | | | |
| PAPAYA BASE | | | | | |
| PARAWOOD BASE | | 0.0003 | | | |
| PEONILE | | | | | |
| PYRALONE | | 0.0060 | | | |
| RHUBAFURAN | | | | | |
| ROSACETOL | | | | | |
| ROSE OIL | | | | | |
| ROSSITOL | | | | | |
| SANDALORE | | | | | |
| SANDALWOOD OIL | | 0.0200 | 0.2000 | | |
| SCENTENAL | | | 0.1000 | | |
| SPIKE LAVENDER OIL | | 0.2000 | | | |
| STEMONE | | | 0.0050 | | |
| SYLKOLIDE | | | | | |
| TERPINYL ACETATE | | | | | |
| THIOGERANIOL | | | | | |
| THYME OIL | | 0.0200 | 0.0200 | | |
| TOLYL ALDEHYDE PARA | | | | | |
| TONKA BEANS EXTRACT | | 0.0200 | | | |
| UNDECAVERTOL | | | 0.1000 | | |
| VANILLA BASE | | 0.2000 | | | |
| VERNALDEHYDE | | | 0.0800 | | |
| VETIVERYL ACETATE | | 0.4000 | 04000 | | |
| YARA YARA | | | | | |
| ZINARINE | | 0.0010 | | | |
| Sum | | 100 | 100 | 100 | 100 |

| | | | | |
|---|---|---|---|---|
| Total % INERT | 12.02 | 49.70 | 19.06 | 38.88 |
| No. of HMP ingredients | 4 | 3 | 0 | 5 |
| Total % HMP ingredients (excl. | 5.59 | 0.82 | 0.00 | 5.98 |
| INERT) | | | | |
| Total % HMI ingredients (excl. | 18.54 | 7.99 | 8.15 | 22.08 |
| INERT) | | | | |
| Total % HMR ingredients (excl. | 11.14 | 6.36 | 42.12 | 10.14 |
| INERT) | | | | |
| Total % IMPU ingredients (excl. | 17.19 | 32.24 | 58.38 | 17.37 |
| INERT) | | | | |
| Total % GEN ingredients (excl. | 37.74 | 33.40 | 30.39 | 33.30 |
| INERT) | | | | |
| Total % RMP ingredients (excl. | 2.18 | 1.79 | 0.00 | 5.79 |
| INERT) | | | | |
| HMPs + HMRs | 16.73 | 7.19 | 42.12 | 16.12 |
| HMPs + HMIs | 24.13 | 8.82 | 8.15 | 28.06 |
| HMPs + HMIs + HMRs + GENs | 73.01 | 48.57 | 80.66 | 71.50 |
| HMPs/(HMPs + RMPs + | 0.22 | 0.02 | 0.00 | 0.21 |
| IMPUs) | | | | |
| HMPs/(HMPs + RMPs + | 0.17 | 0.02 | 0.00 | 0.17 |
| IMPUs + (100-TOTAL)) | | | | |
| RESULT | FAIL | FAIL | FAIL | FAIL |

| | | E | F | G | H |
|---|---|---|---|---|---|
| Ingredient | Group | wt % | wt % | wt % | wt % |
| CEDRYL METHYL ETHER | GEN | | | | |
| CEPIONATE | GEN | | | | |
| HEDIONE | GEN | 15.0000 | | 10.5263 | |
| HEXYL CINNAMIC | GEN | | 3.7209 | | 11.8182 |
| ALDEHYDE | | | | | |
| IONONE BETA | GEN | 0.0400 | | | 0.0545 |
| ISO E SUPER | GEN | | 3.7209 | 2.1053 | |
| LILIAL | GEN | 20.0000 | 7.4419 | | |
| LINALOOL | GEN | 5.5000 | 9.3023 | 6.3158 | 10.0000 |
| PHENYL ETHYL ALCOHOL | GEN | 1.0000 | 1.8605 | 0.2105 | |
| SANDELA | GEN | | 0.1860 | | |
| THIBETOLIDE | GEN | | 0.9302 | | |
| AGRUMEX | HMI | 0.5000 | 11.1628 | 0.7895 | 1.8182 |
| AMBROFIX | HMI | | | 0.0211 | |
| CETALOX | HMI | | | | |
| DAMASCONE ALPHA | HMI | | | | |
| DIHYDRO | HMI | 1.0000 | 1.7209 | | |
| MYRCENOL | | | | | |
| DIMETHYL | HMI | | 0.0140 | 0.3684 | |
| ANTHRANILATE | | | | | |
| EUGENOL | HMI | | | | |
| MANDARIN OIL | HMI | | | | |
| MANDARINAL BASE | HMI | | | 0.4211 | |
| ORANGE OIL | HMI | | 0.4186 | 8.9474 | |
| ORANGE TERPENES | HMI | | 2.7907 | | 5.4545 |
| TRICYCLAL | HMI | 0.2000 | 1.8605 | 0.5263 | 0.1818 |
| TROPIONAL | HMI | 3.0000 | 0.1395 | 0.7895 | |
| ALLYL CAPROATE | HMP | | | 0.7895 | |
| ALLYL CYCLOHEXYL | HMP | | | 0.3684 | |
| PROPIONATE | | | | | |
| ALLYL OENANTHATE | HMP | | | 0.4211 | 0.9091 |
| CALONE FINE | HMP | 0.1000 | | 0.0211 | |
| CASSIS BASE | HMP | 1.7964 | | | |
| DAMASCENONE | HMP | 0.0500 | | 0.0211 | 0.0455 |
| DAMASCONE DELTA | HMP | | 0.4651 | 0.1053 | |
| DECALACTONE | HMP | 0.3000 | 0.0698 | | |
| GAMMA | | | | | |
| DIMETHYL BENZYL | HMP | | | 0.7895 | |
| CARBINYL BUTYRATE | | | | | |
| ETHYL ACETOACETATE | HMP | 0.5000 | | | |
| ETHYL BUTYRATE | HMP | | | 0.1053 | 0.0909 |
| ETHYL ISOBUTYRATE | HMP | | 0.0930 | 0.0526 | 0.0455 |
| ETHYL MALTOL | HMP | | 0.0558 | 0.2632 | 0.3636 |
| GRAPEFRUIT OIL | HMP | | | | 0.2727 |
| HEXENYL-3-CIS | HMP | 2.0000 | | 1.8421 | |
| SALICYLATE | | | | | |
| HEXYL ACETATE | HMP | | 8.8372 | 1.5789 | 0.9091 |
| METHYL CEDRYL KETONE | HMP | | | | |
| METHYL PAMPLEMOUSSE | HMP | | | 0.6316 | |
| STYRALLYL ACETATE | HMP | | 2.7907 | | 2.7273 |
| UNDECAVERTOL | HMP | 4.0000 | 1.6744 | | |
| VERTOFIX COEUR | HMP | | | | |
| BENZYL ACETATE | HMR | 0.5000 | 0.3721 | 4.2105 | |
| CITRONELLOL | HMR | 1.0000 | | 0.7895 | |
| CYCLOHEXAL | HMR | | | | |
| ETHYL LINALOOL | HMR | | | | |
| ETHYLENE BRASSYLATE | HMR | | 1.8605 | 4.9737 | |
| FLOROSA | HMR | | | | |
| GERANIOL | HMR | 3.0000 | | 1.0526 | |
| GERANIUM OIL | HMR | | | | |
| ISOEUGENOL | HMR | | 0.1860 | | |
| ISORALDEINE 70 | HMR | 4.0000 | 2.6047 | | |
| JASMONE CIS | HMR | 0.3000 | | | |
| LEMON OIL | HMR | | 0.0651 | | |
| LEMON TERPENES | HMR | | | | |
| PEACH PURE | HMR | | 1.0047 | 3.1579 | 2.7273 |
| ALLYL AMYL GLYCOLATE | IMPU | 0.2000 | | 0.1579 | |
| BENZYL SALICYLATE | IMPU | | | 1.0526 | 0.4545 |
| BERGAMOT OIL | IMPU | | | | |
| BORNEOL CRYSTALS | IMPU | | 0.1860 | | |
| CAMPHOR | IMPU | | 0.8372 | | |
| CARDAMOM SEED OIL | IMPU | | | | |
| CAROPHYLLENE | IMPU | | | | |
| CITRAL | IMPU | | 0.1860 | 0.5263 | |
| CYCLAMEN ALDEHYDE | IMPU | 1.0000 | | 1.0526 | 1.8182 |
| CYCLOGALBANATE | IMPU | 0.0800 | | 0.0526 | |
| DIMETOL | IMPU | | 1.7209 | | |
| EUCALYPTOL | IMPU | | 1.6744 | | |
| EUCALYPTUS GLOBULUS | IMPU | | 1.1163 | | |
| OIL | | | | | |
| EVERNYL | IMPU | 0.0200 | | | |
| FLORALOZONE | IMPU | | | 0.2632 | 0.9091 |
| FRESKOMENTHE | IMPU | | | | |
| LAVANDIN OIL | IMPU | | 0.5581 | | |
| LAVANDIN BASE | IMPU | | | | |
| LIME OIL | IMPU | 0.4000 | | | |
| LIME OXIDE | IMPU | | | | |
| LIME TERPENES BASE | IMPU | | | | 0.4545 |
| LINALYL ACETATE | IMPU | | 0.6977 | 1.3158 | |
| NUTMEG OIL | IMPU | | | | |
| OLIBANUM BASE | IMPU | | | | |
| PATCHOULI OIL | IMPU | | 0.1860 | | |
| PEPPERMINT OIL | IMPU | | | | |
| PEPPER OIL | IMPU | | | | |
| ROSE OXIDE | IMPU | | | 0.0526 | |
| BUTYL HYDROXY | INERT | 0.2000 | | | 0.9091 |
| TOLUENE | | | | | |
| DIETHYL PHTHALATE | INERT | | | | |
| DIPROPYLENE GLYCOL | INERT | 15.4050 | | 35.2632 | 44.0818 |
| DOWANOL TPM | INERT | | 0.0465 | | |
| TRIETHYL CITRATE | INERT | 0.5425 | 0.1381 | 0.4728 | |
| AUBEPINE PARA CRESOL | RMP | | 0.6512 | | |
| ETHYL VANILLIN | RMP | | 0.0744 | 0.2632 | 0.0909 |
| HABANOLIDE | RMP | | | | |
| HYDROXYCITRONELLAL | RMP | 2.5000 | | | |
| PHENOXY ETHYL | RMP | | | 2.3842 | |
| ISOBUTYRATE | | | | | |
| RADJANOL | RMP | 0.5000 | | | |
| TERPINEOL | RMP | | 0.3256 | | 0.3636 |
| VANILLIN | RMP | | 0.0465 | | |
| ADOXAL | | | | | |
| ALDEHYDE C10 DECYLIC | | | 0.4561 | 0.1053 | 0.2273 |
| ALDEHYDE C110 | | | | 0.3721 | |
| UNDECYLIC | | | | | |
| ALDEHYDE | | | 0.0930 | | |
| C11UNDECYLENIC | | | | | |
| ALDEHYDE C12 LAURIC | | | 0.5581 | 0.0526 | 0.1182 |
| ALDEHYDE C12 MNA | | | 0.5581 | | |
| ALDEHYDE C8 OCTYLIC | | | | | 0.0909 |
| ALDEHYDE C9 NONYLIC | | | | | |
| ALDEHYDE ISO C11 | | | 0.3721 | | |
| AMBERKETAL | | | | | |
| AMBERMAX | | | 0.0465 | | |
| AMBRE BASE | | | | | |
| AMBRETTOLIDE | | | | 0.0526 | |
| AMPHERMATE | | | | | |
| APPLE BASE | | | 0.2047 | | |
| AMYL BUTYRATE | | | | | 0.0909 |
| AMYL SALICYLATE | | | | | |
| BASIL OIL | | | | | |
| BENZALDEHYDE | | | 0.0186 | | 0.9091 |
| BOISAMBRENE FORTE | | | | | |
| BORNYL ACETATE | | | 1.6744 | | |
| BOURGEONAL | | | | 0.3684 | 0.1818 |
| BUTYL ACETATE | | | | 0.0526 | |
| BUTYL BUTYRO LACTATE | | | | | 0.9091 |
| BUTYL CYCLOHEXYL | | | 1.1163 | | |
| ACETATE PARA | | | | | |
| BUTYL QUINOLINE | | | | | |
| SECONDARY | | | | | |
| CALONE | | | | | |
| CARVONE LAEVO | | | | | 0.0091 |
| CASMERAN | | | | | |
| CEDARWOOD OIL | | | | | |
| CINNAMON BARK OIL | | | | | |
| CISTUS OIL | | | | | |
| CITRONELLYL ACETATE | | | | | 0.0909 |
| CITRONELLYL NITRILE | | | 0.1023 | | |
| CLOVE LEAF OIL | | | | | |
| CORPS CASSIS | | | | 0.0026 | |
| COSMONE | | | | | |
| COUMARINE | | | 1.4884 | 0.2632 | |
| CUMINIC ALDEHYDE | | | | | 0.0455 |
| DECALACTONE DELTA | | | | 0.3684 | |
| DIMETHYL BENZYL | | | | | 0.5455 |
| CARBINYL ACETATE | | | | | |
| DIPHENYL OXIDE | | | 1.3023 | | 0.0455 |
| DODECENAL | | | | 0.0263 | |
| EBANOL | | | 0.2047 | | |
| ESTRAGOLE | | 0.0500 | 0.0558 | | |
| ETHYL ACETATE | | | | 0.2632 | |
| ETHYL METHYL-2- | | | 0.6512 | 0.2105 | 0.2727 |
| BUTYRATE | | | | | |
| ETHYL OCTANOATE | | | | | 0.0636 |
| FENNALDEHYDE | | | | | |
| FLORHYDRAL | | | | 0.4211 | 0.4545 |
| FLOROCYCLENE | | | 1.3953 | 0.5263 | |
| FIXOLIDE | | 4.5000 | | | |
| FOLIONE | | 0.0400 | | | |
| FRESCILE | | | | | |
| GALAXOLIDE | | 10.0000 | 4.2047 | | |
| GALBANONE | | | 0.0465 | | |
| GALBANUM OIL | | | | | |
| GALBEX | | | | | |
| GARDOCYCLENE | | | 2.0930 | | |
| GERANYL ACETATE | | | | | |
| GUAVE BASE | | | | | 2.7273 |
| HELIOTROPINE | | | | | |
| HEXENAL-2-TRANS | | | | 0.0105 | |
| HEXENOL-3-CIS | | 0.1000 | | 0.1053 | 0.1818 |
| HEXENYL-3-CIS ACETATE | | | 0.1674 | 0.1579 | |
| HYDROXY ETHYL METHYL | | | | 0.0011 | |
| THIAZOL | | | | | |
| INDOLE | | 0.0050 | | 0.0053 | |
| IRALIA | | | | | |
| IRONE ALPHA | | | | | |
| ISOAMYL ACETATE | | | | 0.2105 | 0.0455 |
| ISOEUGENOL | | 0.1000 | | | |
| ISOPROPYL-2-METHYL-4 | | | | 0.0105 | |
| THIAZOLE | | | | | |
| ISOPROPYL METHYL-2- | | | | | |
| BUTYRATE | | | | | |
| ISORALDEINE CETONE | | | | | |
| ALPHA | | | | | |
| JASMACYCLENE | | | 3.4884 | | 0.9091 |
| JASMALACTONE | | 0.1000 | | | |
| JAVANOL | | | | 0.0263 | |
| KARANAL | | | | | |
| KEPHALIS | | | | | |
| LABIENOXIME | | | 0.0009 | | |
| LAVENDER OIL | | | | | |
| LEMONGRASS OIL | | | | | |
| LEMONILE | | | 0.0465 | | |
| LIFFAROME | | | | 0.0263 | |
| MANGO BASE | | | | | 2.2727 |
| MANZANATE | | 0.0100 | 0.1395 | 0.1053 | |
| MARENIL | | | | | |
| MELONAL | | 0.2000 | | 0.0526 | |
| METHYL ANTHRANILATE | | | 0.0372 | | |
| METHYL CAMOMILLE | | | 0.1860 | | |
| METHYL HEPTENONE | | 0.0300 | | | |
| METHYL-2 BUTANOL-1 | | 0.0200 | | | |
| MUSK KETONE | | | 0.9302 | | |
| MYRALDENE | | | | 0.0105 | |
| MYRCENE 90 | | 0.0036 | | | |
| NECTARYL | | | 0.4651 | | |
| NEOCASPIRENE | | | | | |
| NEROLI OIL | | | | | |
| NONALACTONE GAMMA | | | 0.0465 | 0.3158 | 0.1818 |
| NONENOL-6-CIS | | 0.0005 | | | |
| NYMPHEAL | | | | 0.1579 | |
| OAKMOSS BASE | | | | | |
| ORANGE FLOWER ETHER | | | | | 0.0909 |
| ORANGER CRYSTALS | | | 0.0465 | | |
| ORRIS BASE | | | | | |
| OSYROL | | | | | |
| OXANE | | 0.0025 | | 0.0132 | |
| PAPAYA BASE | | | | | 2.7273 |
| PARAWOOD BASE | | | | | |
| PEONILE | | | 0.1860 | | |
| PYRALONE | | | | | |
| RHUBAFURAN | | | | 0.0526 | |
| ROSACETOL | | | 0.9302 | | |
| ROSE OIL | | 0.2000 | | | |
| ROSSITOL | | | 0.0465 | | |
| SANDALORE | | | | 0.6316 | |
| SANDALWOOD OIL | | | | | |
| SCENTENAL | | | | 0.0789 | |
| SPIKE LAVENDER OIL | | | | | |
| STEMONE | | | | | |
| SYLKOLIDE | | | | 0.2632 | |
| TERPINYL ACETATE | | | | | 0.0364 |
| THIOGERANIOL | | | 0.0005 | | |
| THYME OIL | | | | | |
| TOLYL ALDEHYDE PARA | | | | | 0.2727 |
| TONKA BEANS EXTRACT | | | | | |
| UNDECAVERTOL | | | | | |
| VANILLA BASE | | | | | |
| VERNALDEHYDE | | | | | |
| VETIVERYL ACETATE | | | | | |
| YARA YARA | | | 2.4651 | | |
| ZINARINE | | | | | |
| Sum | | 100 | 100 | 100 | 100 |

| | | | | |
|---|---|---|---|---|
| Total % INERT | 16.15 | 0.18 | 35.74 | 44.99 |
| No. of HMP ingredients | 7 | 7 | 13 | 8 |
| Total % HMP ingredients (excl. | 10.43 | 14.01 | 10.88 | 9.75 |
| INERT) | | | | |
| Total % HMI ingredients (excl. | 5.61 | 18.14 | 18.46 | 13.55 |
| INERT) | | | | |
| Total % HMR ingredients (excl. | 10.49 | 6.10 | 22.07 | 4.96 |
| INERT) | | | | |
| Total % IMPU ingredients (excl. | 2.03 | 6.29 | 6.96 | 6.61 |
| INERT) | | | | |
| Total % GEN ingredients (excl. | 49.54 | 27.21 | 29.81 | 39.76 |
| INERT) | | | | |
| Total % RMP ingredients (excl. | 3.58 | 1.10 | 4.12 | 0.83 |
| INERT) | | | | |
| HMPs + HMRs | 20.93 | 20.12 | 32.95 | 14.71 |
| HMPs + HMIs | 16.04 | 32.15 | 29.34 | 23.30 |
| HMPs + HMIs + HMRs + GENs | 76.07 | 65.47 | 81.22 | 68.02 |
| HMPs/(HMPs + RMPs + | 0.65 | 0.65 | 0.50 | 0.57 |
| IMPUs) | | | | |
| HMPs/(HMPs + RMPs + | 0.30 | 0.29 | 0.37 | 0.23 |
| IMPUs + (100-TOTAL)) | | | | |
| RESULT | PASS | PASS | PASS | PASS |

| | | I | J | K | L |
|---|---|---|---|---|---|
| Ingredient | Group | wt % | wt % | wt % | wt % |
| HEDIONE | GEN | 0.3333 | | 2.0833 | |
| HEXYL CINNAMIC | GEN | 6.6667 | 5.7143 | 8.3333 | |
| ALDEHYDE | | | | | |
| IONONE BETA | GEN | 0.3333 | 0.7619 | 0.8333 | |
| ISO E SUPER | GEN | 1.3333 | | | |
| LILIAL | GEN | | | | |
| LINALOOL | GEN | 10.0000 | 5.7143 | | |
| PHENYL ETHYL ALCOHOL | GEN | | 4.7791 | 6.2500 | |
| SANDELA | GEN | | | | |
| AGRUMEX | HMI | 4.0000 | | 8.3333 | |
| AMBROFIX | HMI | | | 0.0417 | |
| DIHYDRO MYRCENOL | HMI | 3.3333 | 2.8571 | 4.1667 | |
| EUGENOL | HMI | | 0.0952 | 0.0167 | |
| ORANGE OIL | HMI | 4.6667 | 1.4286 | | |
| ORANGE TERPENES | HMI | 20.0000 | | | |
| TRICYCLAL | HMI | 0.5067 | 0.0952 | 0.4167 | |
| TROPIONAL | HMI | 1.3333 | | | |
| ALLYL CYCLOHEXYL | HMP | 0.3333 | | 0.2083 | |
| PROPIONATE | | | | | |
| ALLYL OENANTHATE | HMP | 0.6667 | | | |
| CALONE FINE | HMP | | 0.0571 | 0.1250 | |
| CASSIS BASE | HMP | 0.6667 | 0.1905 | 1.6667 | |
| DAMASCENONE | HMP | | | | |
| DAMASCONE BETA | HMP | | | | |
| DAMASCONE DELTA | HMP | | 0.1905 | 0.1250 | |
| DECALACTONE GAMMA | HMP | | 0.7619 | 1.0000 | 1.2000 |
| DIMETHYL BENZYL | HMP | | | | |
| CARBINYL BUTYRATE | | | | | |
| ESTERLY | HMP | 0.0020 | | | |
| ETHYL BUTYRATE | HMP | 0.0933 | | | |
| ETHYL HEXANOATE | HMP | | | 1.2500 | |
| ETHYL MALTOL | HMP | 0.6667 | | | |
| FLOROPAL | HMP | 1.3333 | | | |
| GRAPEFRUIT OIL | HMP | 0.2000 | | | |
| HEXENYL-3-CIS | HMP | | 1.4286 | | |
| SALICYLATE | | | | | |
| HEXYL ACETATE | HMP | 2.6667 | | 1.1917 | 87.5000 |
| METHYL CEDRYL KETONE | HMP | | 1.9048 | | |
| METHYL PAMPLEMOUSSE | HMP | 0.5333 | | | |
| POMAROSE | HMP | 0.0067 | | | |
| POMELOL | HMP | 1.0667 | | | |
| RASPBERRY KETONE | HMP | 0.1333 | 0.0095 | | |
| STRAWBERRY PURE | HMP | | | | |
| STYRALLYL ACETATE | HMP | 7.6667 | | 0.4167 | |
| UNDECAVERTOL | HMP | 0.2000 | | 1.6667 | |
| VELOUTONE | HMP | | | | 0.1000 |
| YLANG YLANG OIL | HMP | | | | |
| BENZYL ACETATE | HMR | | 1.9048 | 8.3333 | |
| CITRONELLOL | HMR | 2.6667 | | 6.2500 | |
| CYCLOHEXAL | HMR | | | | |
| ETHYL LINALOOL | HMR | | | 10.0000 | |
| ETHYLENE BRASSYLATE | HMR | | 3.8095 | 2.0833 | |
| FLOROSA | HMR | 2.6667 | | 2.0833 | |
| GERANIOL | HMR | | 4.7619 | 1.6667 | |
| GERANIUM OIL | HMR | | | | |
| ISORALDEINE 70 | HMR | | 1.9048 | 2.0833 | |
| ISORALDEINE 95 | HMR | | | | |
| JASMONE CIS | HMR | | 0.0381 | 0.0833 | |
| LEMON OIL | HMR | | | | |
| PEACH PURE | HMR | 1.3333 | | | |
| ALLYL AMYL GLYCOLATE | IMPU | 0.4000 | 0.1905 | 0.4167 | |
| BENZYL SALICYLATE | IMPU | 0.4545 | | | |
| BERGAMOT OIL | IMPU | | | | |
| CAMPHOR | IMPU | | | | |
| CITRAL | IMPU | 0.3333 | | | |
| CYCLAMEN ALDEHYDE | IMPU | 0.6667 | 1.4286 | 0.8333 | |
| CYCLOGALBANATE | IMPU | | 0.1905 | 0.0833 | |
| EUCALYPTUS GLOBULUS | IMPU | | | | |
| OIL | | | | | |
| EVERNYL | IMPU | 0.0200 | | | |
| FLORALOZONE | IMPU | | | 1.2500 | |
| LAVANDIN OIL | IMPU | | | | |
| LINALOOL OXIDE | IMPU | | 0.0381 | | |
| LINALYL ACETATE | IMPU | 0.6667 | | | |
| ROSE OXIDE | IMPU | | 0.0381 | | |
| TRIMOFIX O | IMPU | | | | |
| BENZYL BENZOATE | INERT | | | | |
| DIPROPYLENE GLYCOL | INERT | 7.3733 | 46.9904 | | |
| DOWANOL TPM | INERT | | | 21.1333 | |
| ISOPROPYL MYRISTATE | INERT | | | | |
| TINOGUARD | INERT | | | | |
| TOCOPHEROL ALPHA | INERT | | | | |
| TRIETHYL CITRATE | INERT | 0.5700 | 0.0171 | | |
| AUBEPINE PARA CRESOL | RMP | | | | |
| ETHYL VANILLIN | RMP | | | 0.0417 | |
| HEXYL SALICYLATE | RMP | 5.3333 | | | |
| HYDROXYCITRONELLAL | RMP | | | | |
| JASMIN OIL | RMP | | | | |
| RADJANOL | RMP | | | | |
| TERPINEOL | RMP | | | | |
| VANILLIN | RMP | | | | |
| ACETAL CD | | | | | |
| ACETOIN | | | | | |
| ACETYL ISOEUGENOL | | | | | |
| AGARBOIS | | 0.6667 | | | |
| ALDEHYDE C10 DECYLIC | | 0.2200 | 0.0381 | | |
| ALDEHYDE C11 | | | 0.0190 | | |
| UNDECYLENIC | | | | | |
| ALDEHYDE C12 LAURIC | | 0.0667 | | | |
| ALDEHYDE C12 MNA | | | | 0.0833 | |
| ALDEHYDE C6 HEXYLIC | | | | | |
| ALDEHYDE C8 OCTYLIC | | 0.1667 | | | |
| ALDEHYDE C12 MNA | | | | | |
| ALDEHYDE ISO C11 | | | | | |
| ALDEHYDE MANDARINE | | 0.0033 | | | |
| AMBER XTREME | | | | | |
| AMBERKETAL | | | | | |
| AMBRETTOLIDE | | | | | |
| AMBROCENIDE | | | | | |
| AMYL BUTYRATE | | | | 0.2083 | |
| ANETHOLE | | | 0.0476 | | |
| AZURONE | | | 0.0019 | | |
| BENZALDEHYDE | | 0.0800 | | | 0.1000 |
| BENZYL METHYL ETHER | | | 0.0095 | | |
| BUTYL CYCLOHEXYL | | | 7.6190 | | |
| ACETATE PARA | | | | | |
| CALYPSONE | | 0.0133 | | | |
| CARVONE LAEVO | | 0.2667 | | | |
| CASMERAN | | | | | |
| CEDARWOOD OIL | | | | | |
| CINNAMIC ALCOHOL | | | | | |
| CITRONELLYL ACETATE | | 0.3333 | | 0.8333 | |
| CITRONELLYL NITRILE | | | 0.0190 | | |
| CITRONELLYL | | | 0.0381 | | |
| OXYACETALDEHYDE | | | | | |
| CORPS PAMPLEMOUSSE | | 0.0013 | | | |
| COUMARINE | | | | | |
| CRESYL METHYL ETHER | | | 0.0190 | | |
| PARA | | | | | |
| CUMINIC ALDEHYDE | | 0.0067 | | | |
| DECALACTONE DELTA | | | | | |
| DIETHYL MALONATE | | 1.6667 | | | |
| DIHYDRO EUGENOL | | | 0.3810 | | |
| DIMETHYL BENZYL | | | 1.4286 | | |
| CARBINYL ACETATE | | | | | |
| DIMETHYL PHENYL ETHYL | | | | | |
| CARBINOL | | | | | |
| DIPHENYL OXIDE | | | | | |
| EBANOL | | | | | |
| ETHYL ACETATE | | 0.3333 | | | |
| ETHYL METHYL-2- | | 0.2667 | | 0.0833 | 10.0000 |
| BUTYRATE | | | | | |
| FIXOLIDE | | | | | |
| FLORHYDRAL | | 0.1333 | 0.5714 | | |
| FLORIDILE | | 0.0067 | | | |
| FLOROCYCLENE | | 0.6667 | | | |
| FRUCTONE | | 1.3333 | | 0.8333 | |
| GALAXOLIDE | | 2.0000 | | | |
| GALBANONE | | | 0.0286 | 0.0333 | |
| GERANYL ACETATE | | 0.3333 | | | |
| HELIOTROPINE | | | | | |
| HEXENOL-3-CIS | | 0.1333 | | 0.2500 | 0.6000 |
| HEXENYL-3-CIS ACETATE | | 0.0667 | 0.0952 | 0.2083 | |
| HEXENYL-3-CIS | | | | | |
| ISOBUTYRATE | | | | | |
| HEXYL ISOBUTYRATE | | | | 0.8333 | |
| HOMOFURONOL | | 0.0067 | | | |
| INDOFLOR | | | 0.1905 | | |
| INDOLE | | | | 0.0417 | |
| IRISONE ALPHA | | | | | |
| ISOAMYL ACETATE | | 0.1333 | | | 0.5000 |
| ISOBUTAVAN | | | | | |
| ISOCYCLOCITRAL | | | 0.0095 | | |
| ISOLONGIFOLANONE | | | 0.2857 | | |
| ISOPROPYL METHYL-2- | | | | 0.2083 | |
| BUTYRATE | | | | | |
| JASMALACTONE | | | | | |
| JASMOPYRANE FORTE | | | 0.7619 | | |
| LABIENOXIME | | 0.0033 | | | |
| LEMONILE | | 0.0067 | | | |
| LIFFAROME | | 0.0067 | | | |
| MANZANATE | | 0.5333 | | 0.2500 | |
| MAYOL | | | | | |
| MEFRANAL | | | 0.0095 | | |
| MELONAL | | 0.0667 | 0.0476 | 0.3333 | |
| METHYL ANTHRANILATE | | | | | |
| METHYL HEPTENONE | | | 0.0095 | | |
| METHYL ISOEUGENOL | | | | | |
| MUSK KETONE | | | | | |
| NEROL | | | | | |
| NEROLIDOL | | | | | |
| NEROLINE | | | | | |
| NONALACTONE GAMMA | | 0.2000 | | | |
| NYMPHEAL | | | | 0.8333 | |
| OCTALACTONE GAMMA/ | | | | | |
| DELTA | | | | | |
| OXYOCTALINE FORMATE | | | | | |
| PHENYL ACETALDEHYDE | | | 0.0220 | | |
| PHENYL ETHYL ACETATE | | | | | |
| PHENYL ETHYL PHENYL | | | 0.0952 | | |
| ACETATE | | | | | |
| POLYSANTOL | | | | | |
| PRUNELLA BASE | | | | | |
| RHODINOL | | | | | |
| RHUBAFURAN | | 0.0333 | | | |
| ROSE OIL | | | | | |
| ROSYFOLIA | | | | 0.1667 | |
| ROSYRANE SUPER | | | | | |
| SANDALORE | | | | | |
| SILVIAL | | | 0.9524 | | |
| SPIROGALBANONE | | 0.0067 | | | |
| TERPINYL ACETATE | | 0.6667 | | | |
| TETRAHYDRO LINALOOL | | | | | |
| TRIFERNAL | | | | | |
| VIRIDINE | | | | 0.3333 | |
| Sum | | 100 | 100 | 100 | 100 |
| Total % INERT | | 7.94 | 47.01 | 21.13 | 0.00 |
| No. of HMP ingredients | | 15 | 7 | 9 | 3 |
| Total % HMP ingredients (excl. | | 17.64 | 8.57 | 9.70 | 88.80 |
| INERT) | | | | | |
| Total % HMI ingredients (excl. | | 35.46 | 8.45 | 16.45 | 0.00 |
| INERT) | | | | | |
| Total % HMR ingredients (excl. | | 7.24 | 23.44 | 41.31 | 0.00 |
| INERT) | | | | | |
| Total % IMPU ingredients (excl. | | 2.27 | 3.56 | 3.80 | 0.00 |
| INERT) | | | | | |
| Total % GEN ingredients (excl. | | 20.28 | 32.02 | 22.19 | 0.00 |
| INERT) | | | | | |
| Total % RMP ingredients (excl. | | 5.79 | 0.00 | 0.05 | 0.00 |
| INERT) | | | | | |
| HMPs + HMRs | | 24.88 | 32.01 | 51.01 | 88.80 |
| HMPs + HMIs | | 53.09 | 17.02 | 26.12 | 88.80 |
| HMPs + HMIs + HMRs + GENs | | 80.61 | 72.48 | 89.66 | 88.80 |
| HMPs/(HMPs + RMPs + | | 0.69 | 0.71 | 0.72 | 1.00 |
| IMPUs) | | | | | |
| HMPs/(HMPs + RMPs + | | 0.48 | 0.24 | 0.48 | 0.89 |
| IMPUs + (100-TOTAL)) | | | | | |
| RESULT | | PASS | PASS | PASS | PASS |
| | | M | N | O | P |
| Ingredient | Group | wt % | wt % | wt % | wt % |
| HEDIONE | GEN | 6.7000 | 8.0000 | 24.3739 | 15.0000 |
| HEXYL CINNAMIC | GEN | | | 3.0435 | |
| ALDEHYDE | | | | | |
| IONONE BETA | GEN | | 3.0000 | 1.3043 | 0.5000 |
| ISO E SUPER | GEN | 14.3200 | | 15.6522 | 10.0000 |
| LILIAL | GEN | 4.8000 | | | |
| LINALOOL | GEN | | 3.0000 | 6.0870 | |
| PHENYL ETHYL ALCOHOL | GEN | 0.5200 | 3.0000 | | |
| SANDELA | GEN | | | 0.0870 | |
| AGRUMEX | HMI | | | 1.7391 | |
| AMBROFIX | HMI | | | 0.0174 | 0.2000 |
| DIHYDRO MYRCENOL | HMI | | 5.0000 | | 0.2000 |
| EUGENOL | HMI | 0.1000 | | 0.1000 | |
| ORANGE OIL | HMI | | 2.0000 | | 1.0000 |
| ORANGE TERPENES | HMI | | | 2.4348 | |
| TRICYCLAL | HMI | | | 0.6087 | |
| TROPIONAL | HMI | | | | 0.5000 |
| ALLYL CYCLOHEXYL | HMP | | | 0.0870 | |
| PROPIONATE | | | | | |
| ALLYL OENANTHATE | HMP | | | 0.3478 | |
| CALONE FINE | HMP | | | 0.0174 | |
| CASSIS BASE | HMP | | | 0.3478 | |
| DAMASCENONE | HMP | | | 0.0174 | |
| DAMASCONE BETA | HMP | 0.0300 | | | |
| DAMASCONE DELTA | HMP | 0.0300 | | 0.1739 | |
| DECALACTONE GAMMA | HMP | | | 0.2609 | |
| DIMETHYL BENZYL | HMP | | | 0.2609 | |
| CARBINYL BUTYRATE | | | | | |
| ESTERLY | HMP | | | | |
| ETHYL BUTYRATE | HMP | | | 0.0174 | |
| ETHYL HEXANOATE | HMP | | | | |
| ETHYL MALTOL | HMP | | 0.8000 | 1.7391 | 0.5000 |
| FLOROPAL | HMP | | | | |
| GRAPEFRUIT OIL | HMP | | | | |
| HEXENYL-3-CIS | HMP | | | | 2.0000 |
| SALICYLATE | | | | | |
| HEXYL ACETATE | HMP | | | 0.6087 | 0.0300 |
| METHYL CEDRYL KETONE | HMP | | | | |
| METHYL PAMPLEMOUSSE | HMP | | | 0.0870 | |
| POMAROSE | HMP | | 0.0500 | | |
| POMELOL | HMP | | | | |
| RASPBERRY KETONE | HMP | | | | |
| STRAWBERRY PURE | HMP | | | 0.0870 | |
| STYRALLYL ACETATE | HMP | 0.5000 | | 0.1739 | |
| UNDECAVERTOL | HMP | | | | |
| VELOUTONE | HMP | | | | |
| YLANG YLANG OIL | HMP | | | | 0.1000 |
| BENZYL ACETATE | HMR | 0.5000 | 3.0000 | 0.3478 | 1.0000 |
| CITRONELLOL | HMR | 0.5300 | 3.0000 | 1.3043 | |
| CYCLOHEXAL | HMR | 4.8000 | | | 3.0000 |
| ETHYL LINALOOL | HMR | 0.0200 | 4.0000 | | 10.0000 |
| ETHYLENE BRASSYLATE | HMR | | 5.0000 | | |
| FLOROSA | HMR | | | 3.0435 | 10.0000 |
| GERANIOL | HMR | 0.0600 | | | |
| GERANIUM OIL | HMR | 0.0100 | | | |
| ISORALDEINE 70 | HMR | | 3.0000 | 1.3043 | 1.5000 |
| ISORALDEINE 95 | HMR | 19.5200 | | | |
| JASMONE CIS | HMR | | 0.0500 | 0.0435 | 0.1000 |
| LEMON OIL | HMR | | | 0.2609 | |
| PEACH PURE | HMR | 0.3000 | | 0.3478 | 0.2000 |
| ALLYL AMYL GLYCOLATE | IMPU | | | 0.3478 | |
| BENZYL SALICYLATE | IMPU | 1.6000 | 2.5000 | 0.8696 | 5.0000 |
| BERGAMOT OIL | IMPU | 1.0000 | | | 5.0000 |
| CAMPHOR | IMPU | | 1.0000 | | |
| CITRAL | IMPU | | | | |
| CYCLAMEN ALDEHYDE | IMPU | | 2.0000 | 1.7391 | |
| CYCLOGALBANATE | IMPU | 0.1000 | | 0.0870 | |
| EUCALYPTUS GLOBULUS | IMPU | | 1.0000 | | |
| OIL | | | | | |
| EVERNYL | IMPU | | | 0.4348 | |
| FLORALOZONE | IMPU | | | 0.1739 | |
| LAVANDIN OIL | IMPU | | 0.1500 | | |
| LINALOOL OXIDE | IMPU | | | | |
| LINALYL ACETATE | IMPU | | 3.0000 | 1.7391 | |
| ROSE OXIDE | IMPU | | | | |
| TRIMOFIX O | IMPU | | | | 0.5000 |
| BENZYL BENZOATE | INERT | 1.0000 | | | |
| DIPROPYLENE GLYCOL | INERT | 0.5400 | 10.9500 | 9.5918 | 5.1200 |
| DOWANOL TPM | INERT | | | | |
| ISOPROPYL MYRISTATE | INERT | 8.7000 | | | |
| TINOGUARD | INERT | | | 0.1739 | |
| TOCOPHEROL ALPHA | INERT | | | 0.0870 | |
| TRIETHYL CITRATE | INERT | | | 0.8609 | 0.1980 |
| AUBEPINE PARA CRESOL | RMP | | 3.0000 | 0.0087 | 0.5000 |
| ETHYL VANILLIN | RMP | 0.5000 | 1.5000 | 0.0870 | 0.2000 |
| HEXYL SALICYLATE | RMP | | | 1.7391 | |
| HYDROXYCITRONELLAL | RMP | | | | 1.0000 |
| JASMIN OIL | RMP | 0.3000 | | | |
| RADJANOL | RMP | | | 0.1739 | 1.0000 |
| TERPINEOL | RMP | | 3.0000 | 1.7391 | |
| VANILLIN | RMP | 1.0000 | | | 0.5000 |
| ACETAL CD | | | | 0.3478 | |
| ACETOIN | | | 0.0300 | | |
| ACETYL ISOEUGENOL | | | | | 0.1000 |
| AGARBOIS | | | | | |
| ALDEHYDE C10 DECYLIC | | | 0.5000 | 0.0348 | |
| ALDEHYDE C11 | | | 0.2000 | | |
| UNDECYLENIC | | | | | |
| ALDEHYDE C12 LAURIC | | | 0.2500 | 0.0348 | |
| ALDEHYDE C12 MNA | | | | | |
| ALDEHYDE C6 HEXYLIC | | | | | |
| ALDEHYDE C8 OCTYLIC | | | 0.1000 | | |
| ALDEHYDE C12 MNA | | | 0.8000 | | |
| ALDEHYDE ISO C11 | | | 0.1000 | | |
| ALDEHYDE MANDARINE | | | | | |
| AMBER XTREME | | | | 0.0226 | 0.0300 |
| AMBERKETAL | | | | 0.1304 | |
| AMBRETTOLIDE | | | | 0.1304 | |
| AMBROCENIDE | | | | 0.0030 | |
| AMYL BUTYRATE | | | | | |
| ANETHOLE | | | | | |
| AZURONE | | | | | |
| BENZALDEHYDE | | | 0.5000 | | |
| BENZYL METHYL ETHER | | | | | |
| BUTYL CYCLOHEXYL | | | 3.0000 | | |
| ACETATE PARA | | | | | |
| CALYPSONE | | | | | |
| CARVONE LAEVO | | | | | |
| CASMERAN | | | | 0.0870 | 1.0000 |
| CEDARWOOD OIL | | | 0.1000 | | |
| CINNAMIC ALCOHOL | | 0.8000 | | | |
| CITRONELLYL ACETATE | | | | 0.8696 | |
| CITRONELLYL NITRILE | | | | | |
| CITRONELLYL | | | | | |
| OXYACETALDEHYDE | | | | | |
| CORPS PAMPLEMOUSSE | | | | | |
| COUMARINE | | | 2.0000 | | 0.2000 |
| CRESYL METHYL ETHER | | | | | |
| PARA | | | | | |
| CUMINIC ALDEHYDE | | | | | |
| DECALACTONE DELTA | | | 0.2000 | | |
| DIETHYL MALONATE | | | | | |
| DIHYDRO EUGENOL | | | | 0.0870 | |
| DIMETHYL BENZYL | | 0.5000 | 1.0000 | 0.1739 | |
| CARBINYL ACETATE | | | | | |
| DIMETHYL PHENYL ETHYL | | | | 0.3478 | |
| CARBINOL | | | | | |
| DIPHENYL OXIDE | | | 1.5000 | | |
| EBANOL | | | 1.0000 | | |
| ETHYL ACETATE | | | | | |
| ETHYL METHYL-2- | | | 0.5000 | 0.4348 | |
| BUTYRATE | | | | | |
| FIXOLIDE | | 6.7000 | | | |
| FLORHYDRAL | | | 0.5200 | | |
| FLORIDILE | | | | | |
| FLOROCYCLENE | | | | 0.4348 | |
| FRUCTONE | | | | | |
| GALAXOLIDE | | 17.0100 | | 7.8261 | 20.0000 |
| GALBANONE | | | | | |
| GERANYL ACETATE | | | 1.0000 | | |
| HELIOTROPINE | | 1.9000 | 1.2000 | 0.0087 | 2.0000 |
| HEXENOL-3-CIS | | | 0.4000 | 0.1043 | 0.0200 |
| HEXENYL-3-CIS ACETATE | | | 0.7000 | 0.2609 | |
| HEXENYL-3-CIS | | | | 0.0435 | |
| ISOBUTYRATE | | | | | |
| HEXYL ISOBUTYRATE | | | | 0.0870 | |
| HOMOFURONOL | | | | | |
| INDOFLOR | | | | | |
| INDOLE | | | | 0.0087 | 0.0020 |
| IRISONE ALPHA | | 1.0000 | 1.0000 | | |
| ISOAMYL ACETATE | | | | 0.0174 | |
| ISOBUTAVAN | | | 0.1000 | | |
| ISOCYCLOCITRAL | | | | | |
| ISOLONGIFOLANONE | | | | | |
| ISOPROPYL METHYL-2- | | | | | |
| BUTYRATE | | | | | |
| JASMALACTONE | | | | | 0.1000 |
| JASMOPYRANE FORTE | | | | | |
| LABIENOXIME | | | | | |
| LEMONILE | | | | | |
| LIFFAROME | | | | 0.0435 | |
| MANZANATE | | | | 0.1739 | |
| MAYOL | | | | 0.8696 | |
| MEFRANAL | | | | | |
| MELONAL | | | | | |
| METHYL ANTHRANILATE | | | 0.2000 | 0.0435 | 0.5000 |
| METHYL HEPTENONE | | | | | |
| METHYL ISOEUGENOL | | | | 0.1739 | |
| MUSK KETONE | | 0.5000 | | | |
| NEROL | | 0.0400 | | | |
| NEROLIDOL | | 0.0100 | | | |
| NEROLINE | | | 0.5000 | | |
| NONALACTONE GAMMA | | | 6.5000 | | 0.1000 |
| NYMPHEAL | | | 0.5000 | 0.8696 | |
| OCTALACTONE GAMMA/ | | | 1.2000 | | |
| DELTA | | | | | |
| OXYOCTALINE FORMATE | | 0.4000 | | | |
| PHENYL ACETALDEHYDE | | | | | |
| PHENYL ETHYL ACETATE | | | 0.1000 | | |
| PHENYL ETHYL PHENYL | | | | 0.0783 | |
| ACETATE | | | | | |
| POLYSANTOL | | | | | 1.0000 |
| PRUNELLA BASE | | 2.0000 | | | |
| RHODINOL | | 0.2500 | | | |
| RHUBAFURAN | | | | | |
| ROSE OIL | | 0.0100 | | | |
| ROSYFOLIA | | | | | 0.1000 |
| ROSYRANE SUPER | | | 0.1000 | | |
| SANDALORE | | 1.4000 | | 0.0870 | |
| SILVIAL | | | | | |
| SPIROGALBANONE | | | | | |
| TERPINYL ACETATE | | | | | |
| TETRAHYDRO LINALOOL | | | 3.0000 | | |
| TRIFERNAL | | | 0.2000 | | |
| VIRIDINE | | | | | |
| Sum | | 100 | 100 | 100 | 100 |

| | | | | |
|---|---|---|---|---|
| Total % INERT | 10.24 | 10.95 | 10.71 | 5.32 |
| No. of HMP ingredients | 3 | 2 | 12 | 4 |
| Total % HMP ingredients (excl. | 0.62 | 0.95 | 4.83 | 2.78 |
| INERT) | | | | |
| Total % HMI ingredients (excl. | 0.11 | 7.86 | 5.38 | 2.01 |
| INERT) | | | | |
| Total % HMR ingredients (excl. | 28.68 | 20.27 | 7.45 | 27.25 |
| INERT) | | | | |
| Total % IMPU ingredients (excl. | 1.89 | 10.84 | 6.04 | 11.09 |
| INERT) | | | | |
| Total % GEN ingredients (excl. | 29.34 | 19.09 | 56.61 | 26.93 |
| INERT) | | | | |
| Total % RMP ingredients (excl. | 2.01 | 8.42 | 4.20 | 3.38 |
| INERT) | | | | |
| HMPs + HMRs | 29.30 | 21.22 | 12.28 | 30.03 |
| HMPs + HMIs | 0.74 | 8.82 | 10.21 | 4.78 |
| HMPs + HMIs + HMRs + GENs | 58.76 | 48.18 | 74.27 | 58.97 |
| HMPs/(HMPs + RMPs + | 0.14 | 0.05 | 0.32 | 0.16 |
| IMPUs) | | | | |
| HMPs/(HMPs + RMPs + | 0.01 | 0.02 | 0.16 | 0.06 |
| IMPUs + (100-TOTAL)) | | | | |
| RESULT | FAIL | FAIL | FAIL | FAIL |

### EXAMPLE 4: RESULTS OF FUNCTIONAL NEAR INFRARED SPECTROSCOPY TESTING

fNIRS testing of fragrance compositions A through P described in Example 4 was conducted according to the method described in Example 1. A non-odour control was used as the benchmark.

As a first level, conditions A1 through A6 and B1 through B10 were investigated:
A1. Deoxy Hb for the full brain shows a statistically significant decrease after 30 seconds of smelling;
A2. Deoxy Hb for the right brain hemisphere shows a statistically significant decrease after 30 seconds of smelling;
A3. Total Hb for the full brain shows a statistically significant decrease after 30 seconds of smelling;
A4. Total Hb for the right brain hemisphere shows a statistically significant decrease after 30 seconds of smelling;
A5. Deoxy Hb for the right brain hemisphere shows a statistically significant increase after 0-10 seconds of smelling;
A6. Oxy Hb for the right brain hemisphere shows a statistically significant increase after 5-10 seconds of smelling;
B1. Channel 3 shows a statistically significant increase of Deoxy Hb after 30 seconds of smelling;
B2. Channel 20 shows a statistically significant increase of Deoxy Hb after 30 seconds of smelling;
B3. Channel 12 shows a statistically significant decrease of Total Hb after 30 seconds of smelling;
B4. Channel 5 shows a statistically significant increase of Oxy Hb after 0-5 seconds of smelling;
B5. Channel 4 shows a statistically significant increase of Total Hb after 0-5 seconds of smelling;
B6. Channel 5 shows a statistically significant increase of Total Hb after 0-5 seconds of smelling;
B7. Channel 11 shows a statistically significant increase of Oxy Hb after 0-10 seconds of smelling;
B8. Channel 8 shows a statistically significant decrease of Deoxy Hb after 5-10 seconds of smelling;
B9. Channel 10 shows a statistically significant decrease of Deoxy Hb after 5-10 seconds of smelling;
B10. Channel 18 shows a statistically significant increase of Oxy Hb after 5-10 seconds of smelling.

Based on extensive testing, it had been determined that at least three out of the six conditions A1 through A6 (Criterion A) and/or at least five out of the ten conditions B1 through B10 (Criterion B) are met in case a fragrance composition provides a happy effect.

The results of the first level INIRS testing are shown in the following two tables:

| Condition | | A | B | C | D | E | F | G | H |
|---|---|---|---|---|---|---|---|---|---|
| A1 | Test Fragrance | −0.113 | 0.001 | −0.018 | 0.031 | −0.031 | −0.130 | −0.019 | 0.012 |
| | Benchmark | −0.027 | −0.001 | 0.055 | 0.004 | −0.001 | 0.556 | 0.072 | −0.060 |
| | Condition met | Yes | No | Yes | No | Yes | Yes | Yes | No |
| A2 | Test Fragrance | −0.103 | −0.028 | 0.024 | 0.020 | −0.034 | −0.136 | −0.017 | 0.045 |
| | Benchmark | −0.006 | 0.005 | 0.042 | −0.004 | 0.005 | 0.049 | 0.019 | −0.078 |
| | Condition met | Yes | Yes | No | No | Yes | Yes | Yes | No |
| A3 | Test Fragrance | −0.027 | −0.005 | −0.078 | 0.036 | 0.055 | −0.027 | −0.043 | −0.113 |
| | Benchmark | 0.067 | 0.017 | 0.015 | −0.056 | 0.017 | 0.031 | 0.063 | 0.020 |
| | Condition met | Yes | No | Yes | No | No | Yes | Yes | Yes |
| A4 | Test Fragrance | −0.067 | −0.058 | −0.089 | 0.022 | 0.071 | −0.117 | −0.064 | −0.134 |
| | Benchmark | 0.096 | 0.035 | 0.030 | −0.057 | 0.035 | 0.049 | 0.021 | 0.012 |
| | Condition met | Yes | Yes | Yes | No | No | Yes | Yes | Yes |
| A5 | Test Fragrance | −0.164 | −0.030 | −0.117 | −0.073 | 0.037 | −0.040 | −0.026 | −0.002 |
| | Benchmark | −0.014 | 0.008 | 0.092 | −0.057 | 0.008 | 0.142 | 0.058 | −0.062 |
| | Condition met | No | No | No | No | Yes | No | No | Yes |
| A6 | Test Fragrance | −0.008 | −0.149 | −0.149 | 0.075 | −0.068 | −0.266 | −0.015 | −0.043 |
| | Benchmark | 0.08 | −0.108 | −0.108 | −0.110 | −0.108 | 0.003 | −0.040 | −0.015 |
| | Condition met | No | No | No | Yes | Yes | No | No | No |
| Number of conditions | | 4 | 2 | 3 | 1 | 4 | 4 | 4 | 3 |
| A1-A6 met | | | | | | | | | |
| B1 | Test Fragrance | −0.106 | 0.015 | 0.060 | 0.010 | 0.069 | −0.082 | −0.070 | −0.017 |
| | Benchmark | −0.098 | 0.011 | 0.122 | −0.089 | 0.011 | 0.122 | 0.080 | −0.197 |
| | Condition met | No | No | No | Yes | Yes | No | No | Yes |
| B2 | Test Fragrance | −0.003 | 0.033 | 0.031 | 0.084 | −0.021 | −0.007 | −0.080 | 0.020 |
| | Benchmark | 0.010 | −0.062 | −0.066 | −0.085 | −0.062 | −0.066 | 0.043 | −0.280 |
| | Condition met | No | Yes | Yes | Yes | Yes | Yes | No | Yes |
| B3 | Test Fragrance | 0.088 | 0.089 | −0.120 | −0.107 | 0.043 | 0.152 | 0.042 | −0.035 |
| | Benchmark | 0.044 | −0.142 | −0.002 | 0.113 | −0.142 | −0.002 | 0.239 | 0.098 |
| | Condition met | No | No | Yes | Yes | No | No | Yes | Yes |
| B4 | Test Fragrance | 0.183 | 0.047 | 0.060 | 0.065 | −0.070 | 0.096 | 0.109 | 0.095 |
| | Benchmark | 0.099 | −0.183 | 0.001 | 0.092 | −0.183 | 0.001 | 0.096 | 0.192 |
| | Condition met | Yes | Yes | Yes | No | Yes | Yes | No | No |
| B5 | Test Fragrance | 0.018 | 0.236 | 0.127 | 0.419 | 0.098 | 0.059 | 0.196 | 0.003 |
| | Benchmark | 0.118 | −0.037 | −0.148 | 0.228 | −0.037 | −0.148 | 0.135 | −0.175 |
| | Condition met | No | Yes | Yes | Yes | Yes | Yes | Yes | Yes |
| B6 | Test Fragrance | 0.141 | 0.021 | 0.022 | 0.072 | −0.056 | 0.089 | 0.136 | 0.007 |
| | Benchmark | 0.052 | −0.102 | −0.018 | 0.053 | −0.102 | −0.018 | 0.011 | 0.164 |
| | Condition met | Yes | Yes | Yes | No | No | Yes | Yes | No |
| B7 | Test Fragrance | −0.022 | 0.054 | −0.102 | 0.215 | 0.090 | 0.079 | 0.158 | 0.067 |
| | Benchmark | 0.035 | −0.136 | −0.112 | 0.249 | −0.136 | −0.112 | 0.038 | 0.017 |
| | Condition met | No | Yes | No | No | Yes | Yes | Yes | Yes |
| B8 | Test Fragrance | −0.273 | 0.085 | −0.095 | 0.037 | 0.035 | 0.252 | −0.113 | 0.125 |
| | Benchmark | −0.020 | 0.087 | −0.025 | 0.116 | 0.087 | −0.025 | 0.291 | 0.155 |
| | Condition met | Yes | No | Yes | Yes | Yes | No | Yes | Yes |
| B9 | Test Fragrance | −0.277 | 0.041 | −0.125 | −0.069 | −0.152 | −0.115 | 0.170 | −0.154 |
| | Benchmark | 0.094 | 0.303 | 0.135 | 0.125 | 0.303 | 0.135 | 0.054 | 0.345 |
| | Condition met | Yes | Yes | Yes | Yes | Yes | Yes | No | Yes |
| B10 | Test Fragrance | 0.096 | −0.230 | −0.113 | 0.256 | 0.077 | −0.249 | 0.043 | −0.032 |
| | Benchmark | 0.092 | −0.037 | 0.051 | −0.179 | −0.037 | 0.051 | −0.147 | 0.097 |
| | Condition met | No | No | No | Yes | Yes | No | Yes | No |

| | | | | | | | | |
|---|---|---|---|---|---|---|---|---|
| Number of conditions | 4 | 6 | 7 | 7 | 8 | 6 | 6 | 7 |
| B1-B10 met | | | | | | | | |
| Happy effect? | YES | YES | YES | YES | YES | YES | YES | YES |

| Condition | | I | J | K | L | M | N | O | P |
|---|---|---|---|---|---|---|---|---|---|
| A1 | Test Fragrance | 0.008 | −0.007 | −0.087 | −0.065 | 0.034 | −0.043 | −0.005 | 0.005 |
| | Benchmark | 0.023 | 0.004 | 0.023 | 0.052 | −0.027 | −0.026 | −0.011 | 0.004 |
| | Condition met | Yes | Yes | Yes | Yes | No | No | No | No |
| A2 | Test Fragrance | 0.034 | 0.031 | −0.124 | −0.061 | 0.004 | 0.002 | −0.034 | −0.012 |
| | Benchmark | 0.035 | −0.004 | 0.035 | 0.083 | −0.006 | −0.017 | 0.012 | −0.004 |
| | Condition met | No | No | Yes | Yes | No | No | Yes | No |
| A3 | Test Fragrance | 0.001 | 0.038 | 0.112 | 0.103 | 0.035 | 0.017 | 0.005 | 0.003 |
| | Benchmark | −0.092 | −0.057 | −0.092 | 0.075 | 0.067 | 0.023 | −0.066 | −0.056 |
| | Condition met | No | No | No | No | Yes | No | No | No |
| A4 | Test Fragrance | 0.036 | 0.061 | 0.065 | 0.079 | 0.039 | 0.008 | 0.014 | −0.017 |
| | Benchmark | −0.075 | −0.058 | −0.075 | 0.120 | 0.096 | 0.052 | −0.074 | −0.057 |
| | Condition met | No | No | No | Yes | Yes | Yes | No | No |
| A5 | Test Fragrance | −0.069 | −0.047 | −0.156 | −0.111 | −0.048 | 0.045 | −0.058 | −0.044 |
| | Benchmark | −0.091 | −0.057 | −0.091 | 0.118 | −0.014 | 0.014 | −0.071 | −0.057 |
| | Condition met | Yes | No | No | No | No | Yes | No | No |
| A6 | Test Fragrance | 0.070 | −0.014 | 0.069 | 0.102 | −0.013 | 0.085 | −0.032 | 0.002 |
| | Benchmark | −0.140 | −0.110 | −0.140 | 0.033 | 0.084 | 0.030 | −0.088 | −0.110 |
| | Condition met | Yes | Yes | Yes | Yes | No | Yes | Yes | Yes |
| Number of conditions | | 3 | 2 | 3 | 4 | 2 | 3 | 2 | 1 |
| A1-A6 met | | | | | | | | | |
| B1 | Test Fragrance | 0.028 | 0.037 | −0.135 | −0.149 | 0.010 | 0.050 | 0.010 | 0.111 |
| | Benchmark | −0.045 | −0.089 | −0.045 | 0.114 | −0.098 | −0.028 | 0.112 | −0.089 |
| | Condition met | Yes | Yes | No | No | Yes | Yes | No | Yes |
| B2 | Test Fragrance | 0.113 | 0.140 | −0.030 | −0.058 | −0.005 | −0.109 | −0.182 | 0.068 |
| | Benchmark | −0.079 | −0.085 | −0.079 | 0.015 | 0.010 | 0.026 | 0.172 | −0.085 |
| | Condition met | Yes | Yes | Yes | No | No | No | No | Yes |
| B3 | Test Fragrance | −0.172 | −0.052 | 0.209 | 0.148 | −0.040 | 0.144 | −0.014 | 0.063 |
| | Benchmark | −0.020 | 0.113 | −0.020 | 0.007 | 0.044 | 0.005 | −0.060 | 0.113 |
| | Condition met | Yes | Yes | No | No | Yes | No | No | No |
| B4 | Test Fragrance | 0.298 | 0.196 | 0.428 | 0.257 | 0.113 | 0.431 | 0.165 | 0.173 |
| | Benchmark | −0.017 | 0.092 | −0.017 | 0.069 | 0.099 | 0.414 | 0.309 | 0.092 |
| | Condition met | Yes | Yes | Yes | Yes | No | No | No | Yes |
| B5 | Test Fragrance | −0.063 | 0.478 | 0.214 | 0.130 | 0.166 | 0.303 | 0.218 | 0.116 |
| | Benchmark | −0.159 | 0.228 | −0.159 | −0.113 | 0.118 | 0.314 | 0.165 | 0.228 |
| | Condition met | Yes | Yes | Yes | Yes | No | No | Yes | No |
| B6 | Test Fragrance | 0.292 | 0.195 | 0.469 | 0.144 | 0.068 | 0.442 | 0.241 | 0.106 |
| | Benchmark | −0.075 | 0.053 | −0.075 | 0.027 | 0.052 | 0.403 | 0.318 | 0.053 |
| | Condition met | Yes | Yes | Yes | Yes | No | No | No | Yes |
| B7 | Test Fragrance | 0.161 | 0.173 | 0.273 | 0.464 | 0.084 | 0.362 | 0.294 | 0.126 |
| | Benchmark | −0.239 | 0.249 | −0.239 | 0.019 | 0.035 | 0.251 | 0.187 | 0.249 |
| | Condition met | Yes | No | Yes | Yes | Yes | Yes | No | No |
| B8 | Test Fragrance | −0.168 | −0.258 | −0.197 | −0.143 | 0.077 | −0.258 | 0.078 | −0.021 |
| | Benchmark | 0.055 | 0.116 | 0.055 | 0.317 | −0.020 | −0.068 | −0.191 | 0.116 |
| | Condition met | Yes | Yes | Yes | Yes | No | Yes | No | Yes |
| B9 | Test Fragrance | 0.149 | 0.026 | −0.171 | −0.029 | −0.077 | −0.136 | −0.148 | −0.138 |
| | Benchmark | −0.022 | 0.125 | −0.022 | 0.280 | 0.094 | −0.050 | −0.295 | 0.125 |
| | Condition met | Yes | Yes | Yes | Yes | Yes | Yes | No | Yes |
| B10 | Test Fragrance | 0.268 | −0.127 | 0.168 | 0.057 | 0.084 | 0.068 | −0.050 | 0.203 |
| | Benchmark | −0.119 | −0.179 | −0.119 | 0.007 | 0.092 | −0.043 | −0.076 | −0.179 |
| | Condition met | Yes | Yes | Yes | Yes | No | Yes | No | Yes |

| | | | | | | | | |
|---|---|---|---|---|---|---|---|---|
| Number of conditions | 10 | 9 | 8 | 7 | 4 | 5 | 1 | 7 |
| B1-B10 met | | | | | | | | |
| Happy effect? | YES | YES | YES | YES | NO | YES | NO | YES |

For those compositions that fulfilled at least one of Criterion A and Criterion B (Compositions A-L, N and P), a further investigation of specific fNIRS channels and time points was conducted. Specifically, it was tested if any of the following further conditions C1 through C10 were met:
C1. Channel 4 shows a statistically significant increase of Oxy Hb after 30 seconds of smelling;
C2. Channel 4 shows a statistically significant increase of Total Hb after 30 seconds of smelling;
C3. Channel 19 shows a statistically significant increase of Total Hb after 30 seconds of smelling;
C4. Channel 2 shows a statistically significant decrease of Deoxy Hb after 0-5 seconds of smelling;
C5. Channel 8 shows a statistically significant decrease of Deoxy Hb after 0-5 seconds of smelling;
C6. Channel 6 shows a statistically significant increase of Oxy Hb after 0-5 seconds of smelling;
C7. Channel 12 shows a statistically significant decrease of Oxy Hb after 0-5 seconds of smelling;
C8. Channel 13 shows a statistically significant increase of Oxy Hb after 0-5 seconds of smelling;
C9. Channel 16 shows a statistically significant increase of Oxy Hb after 0-5 seconds of smelling;
C10. Channel 6 shows a statistically significant increase of Oxy Hb after 5-10 seconds of smelling.

It was found that fragrance compositions led to a more pronounced improvement of the happiness state if at least at least ten out of the 20 conditions B1 through B10 and C1 through C10 were met (Criterion C).

The results of this second level fNIRS testing are shown in the following two tables:

| Condition | | A | B | C | D | E | F | G |
|---|---|---|---|---|---|---|---|---|
| Number of conditions | | 4 | 6 | 7 | 7 | 8 | 6 | 6 |
| B1-B10 met | | | | | | | | |
| C1 | Test Fragrance | 0.028 | 0.043 | −0.074 | −0.016 | 0.006 | −0.059 | −0.021 |
| | Benchmark | 0.038 | −0.051 | 0.043 | 0.034 | −0.051 | 0.043 | 0.168 |
| | Condition met | No | Yes | No | No | Yes | No | No |
| C2 | Test Fragrance | −0.013 | 0.041 | 0.037 | −0.021 | 0.038 | −0.084 | 0.033 |
| | Benchmark | 0.009 | −0.074 | 0.034 | 0.048 | −0.074 | 0.034 | 0.121 |
| | Condition met | No | Yes | No | No | Yes | No | No |
| C3 | Test Fragrance | 0.080 | −0.074 | −0.181 | −0.063 | 0.050 | −0.047 | −0.158 |
| | Benchmark | 0.043 | −0.083 | −0.021 | 0.036 | −0.083 | −0.021 | 0.077 |
| | Condition met | Yes | No | No | No | Yes | No | No |
| C4 | Test Fragrance | 0.181 | 0.293 | 0.130 | 0.101 | −0.092 | −0.111 | −0.135 |
| | Benchmark | −0.043 | −0.093 | 0.092 | −0.035 | −0.093 | 0.092 | 0.306 |
| | Condition met | No | No | No | No | No | Yes | Yes |
| C5 | Test Fragrance | 0.059 | 0.173 | −0.184 | 0.071 | −0.006 | 0.199 | −0.077 |
| | Benchmark | −0.113 | 0.234 | 0.008 | 0.084 | 0.234 | 0.008 | 0.178 |
| | Condition met | No | Yes | Yes | No | Yes | No | Yes |
| C6 | Test Fragrance | 0.007 | −0.083 | −0.212 | 0.196 | −0.341 | −0.273 | −0.019 |
| | Benchmark | −0.042 | −0.251 | −0.474 | −0.011 | −0.251 | −0.474 | −0.035 |
| | Condition met | No | Yes | Yes | Yes | No | Yes | No |
| C7 | Test Fragrance | 0.550 | 0.166 | −0.061 | 0.374 | −0.225 | −0.185 | 0.285 |
| | Benchmark | 0.374 | −0.123 | −0.295 | 0.462 | −0.123 | −0.295 | 0.405 |
| | Condition met | No | No | No | Yes | Yes | No | Yes |
| C8 | Test Fragrance | 0.169 | 0.176 | −0.076 | 0.428 | 0.025 | −0.025 | 0.312 |
| | Benchmark | −0.051 | −0.120 | −0.242 | 0.344 | −0.120 | −0.242 | 0.283 |
| | Condition met | Yes | Yes | Yes | Yes | Yes | Yes | No |
| C9 | Test Fragrance | 0.256 | −0.032 | −0.129 | −0.043 | 0.041 | 0.010 | 0.056 |
| | Benchmark | −0.119 | 0.070 | −0.127 | −0.140 | 0.070 | −0.127 | 0.207 |
| | Condition met | Yes | No | No | Yes | No | Yes | No |
| C10 | Test Fragrance | 0.042 | −0.224 | −0.082 | 0.124 | −0.302 | −0.107 | 0.082 |
| | Benchmark | 0.079 | −0.210 | −0.129 | −0.142 | −0.210 | −0.129 | 0.024 |
| | Condition met | No | No | No | Yes | No | No | Yes |

| | | | | | | | |
|---|---|---|---|---|---|---|---|
| Number of conditions | 3 | 5 | 3 | 5 | 6 | 4 | 4 |
| C1-C10 met | | | | | | | |
| Criterion C fulfilled? | NO | YES | YES | YES | YES | YES | YES |

| Condition | | H | I | J | K | L | N | P |
|---|---|---|---|---|---|---|---|---|
| Number of conditions | | 7 | 10 | 9 | 8 | 7 | 5 | 7 |
| B1-B10 met | | | | | | | | |
| C1 | Test Fragrance | 0.229 | 0.390 | 0.165 | 0.148 | 0.193 | 0.007 | −0.134 |
| | Benchmark | −0.139 | −0.162 | 0.034 | −0.162 | 0.068 | −0.008 | 0.034 |
| | Condition met | Yes | Yes | Yes | Yes | Yes | No | No |
| C2 | Test Fragrance | 0.202 | 0.033 | 0.133 | 0.137 | 0.187 | 0.008 | −0.078 |
| | Benchmark | −0.140 | −0.183 | 0.048 | −0.183 | 0.054 | −0.023 | 0.048 |
| | Condition met | Yes | Yes | Yes | Yes | Yes | No | No |
| C3 | Test Fragrance | 0.034 | 0.109 | 0.183 | −0.049 | 0.209 | −0.041 | −0.034 |
| | Benchmark | −0.048 | −0.157 | 0.036 | −0.157 | 0.025 | 0.069 | 0.036 |
| | Condition met | Yes | Yes | Yes | Yes | Yes | No | No |
| C4 | Test Fragrance | −0.018 | −0.338 | −0.257 | −0.336 | 0082 | −0.191 | −0.054 |
| | Benchmark | 0.065 | −0.083 | −0.035 | −0.083 | −0.021 | −0.214 | −0.035 |
| | Condition met | Yes | Yes | Yes | Yes | No | No | No |
| C5 | Test Fragrance | 0.119 | −0.005 | −0.316 | −0.238 | −0.078 | −0.080 | −0.213 |
| | Benchmark | 0.142 | 0.030 | 0.084 | 0.030 | 0.243 | 0.002 | 0.084 |
| | Condition met | No | Yes | Yes | Yes | Yes | Yes | Yes |
| C6 | Test Fragrance | 0.053 | 0.183 | 0.137 | 0.247 | 0.068 | −0.108 | 0.056 |
| | Benchmark | −0.065 | −0.131 | −0.011 | −0.131 | 0.137 | 0.014 | −0.011 |
| | Condition met | Yes | Yes | Yes | Yes | No | No | No |
| C7 | Test Fragrance | 0.085 | 0.068 | 0.262 | 0.190 | 0.296 | 0.140 | 0.505 |
| | Benchmark | 0.239 | 0.045 | 0.462 | 0.045 | −0.027 | 0.169 | 0.462 |
| | Condition met | Yes | No | Yes | No | No | No | No |
| C8 | Test Fragrance | 0.086 | 0.210 | 0.472 | 0.110 | 0.365 | 0.073 | 0.279 |
| | Benchmark | 0.243 | −0.003 | 0.344 | −0.003 | 0.213 | 0.122 | 0.344 |
| | Condition met | No | Yes | Yes | Yes | Yes | No | No |
| C9 | Test Fragrance | 0.162 | 0.376 | 0.029 | 0.349 | 0.392 | 0.406 | −0.115 |
| | Benchmark | 0.273 | −0.048 | −0.140 | −0.048 | 0.351 | 0.374 | −0.140 |
| | Condition met | No | Yes | Yes | Yes | No | No | No |
| C10 | Test Fragrance | −0.028 | 0.114 | −0.012 | 0.121 | −0.011 | 0.044 | −0.102 |
| | Benchmark | −0.125 | −0.145 | −0.142 | −0.145 | −0.174 | 0.073 | −0.142 |
| | Condition met | Yes | Yes | Yes | Yes | Yes | No | No |

| | | | | | | | |
|---|---|---|---|---|---|---|---|
| Number of conditions | 7 | 9 | 10 | 9 | 6 | 1 | 1 |
| C1-C10 met | | | | | | | |
| Criterion C fulfilled? | YES | YES | YES | YES | YES | NO | NO |

An even better distinction between the fragrance compositions was found to be possible if also Criterion D was assessed, which requires:
that at least 16, more preferably at least 17, and most preferably at least 18, out of the 20 conditions B1 through B10 and C1 through C10 are met if zero or one out of the conditions A1 through A6 are met;
that at least 12, more preferably at least 13, and most preferably at least 14, out of the 20 conditions B1 through B10 and C1 through C10 are met if two or three out of the conditions A1 through A6 are met; and
that at least 8, more preferably at least 9, and most preferably at least 10, out of the 20 conditions B1 through B10 and C1 through C10 are met if four, five or six out of the conditions A1 through A6 are met.

Criterion D was only applied to those compositions that fulfilled Criterion C (Compositions B-L). The results are shown in the following table:

| | B | C | D | E | F | G | H | I | J | K | L |
|---|---|---|---|---|---|---|---|---|---|---|---|
| Number of conditions | 2 | 3 | 1 | 4 | 4 | 4 | 3 | 3 | 2 | 3 | 4 |
| A1-A6 met | | | | | | | | | | | |
| Number of conditions | 6 | 7 | 7 | 8 | 6 | 6 | 7 | 10 | 9 | 8 | 7 |
| B1-B10 met | | | | | | | | | | | |
| Number of conditions | 5 | 3 | 5 | 6 | 4 | 4 | 7 | 9 | 10 | 9 | 6 |
| C1-C10 met | | | | | | | | | | | |
| Criterion D fulfilled? | NO | NO | NO | YES | YES | YES | YES | YES | YES | YES | YES |

It has been found that the additional Criteria C and D lead to an improved accuracy for predicting the effect on happiness achieved by the fragrance compositions. Consequently, the rules for preparing the fragrance compositions of the invention were devised such that the respective fragrance compositions pass even the highest level of fNIRS testing, i.e. Criterion D.

Furthermore, fNIRS testing shows very specific brain signatures at both group level (i.e. full brain and/or hemispherical averages) and at single channel level, making the validation test so thorough that only fragrance compositions and fragrance ingredients truly providing a sense of happiness in the participant can pass it.

Thus, the compositions of the present invention were found to provide a happy benefit on the sub-conscious level.

### EXAMPLE 5: RESULTS FROM QUESTIONNAIRE

The following table shows an example for results obtained with the questionnaire used in Example 2 (15 participants). Composition J of Example 3 is the test fragrance; a non-odour control was used as the benchmark; and Fragrance 1, 2, and 3 are, respectively, compositions D, N and P of Example 3.

| | | | | Invigo- | Relax- | |
|---|---|---|---|---|---|---|
| | Compo- | Liking | Strength | rating | ing | Happy |
| Sample | sition | (1-9) | (1-9) | (1-9) | (1-9) | (1-9) |
| Test | J | 6.600 | 5.467 | 5.133 | 5.200 | 5.267 |
| Fragrance 1 | D | 6.667 | 6.933 | 5.800 | 4.533 | 5.467 |
| Fragrance 2 | N | 5.600 | 6.733 | 5.000 | 4.333 | 4.800 |
| Fragrance 3 | P | 6.467 | 5.600 | 5.200 | 5.067 | 5.400 |
| Benchmark | — | 3.800 | 1.667 | 1.267 | 1.800 | 1.733 |

It is worth mentioning that the outlined Happy effect at brain level does not depend on the liking of the fragrances: paired t-tests run on Liking scores between the test fragrance and all the other fragrance compositions tested were non-significant, with all p values being larger than 0.15. Therefore, the Happy effect is exclusively due to the composition and not the hedonic character of the fragrance. Furthermore, the data shown in the above table highlight the limitations of consumer tests at differentiating moods based solely on explicit, declarative responses. In fact, there is no significant difference between mood ratings of the same fragrance and mood ratings across the fragrances tested. The only significant difference that has been highlighted regards the presence or absence of fragrance, which is unrelated to the specific mood of the fragrance.

### EXAMPLE 6: RESULTS OF MOOD PORTRAITS® TESTING

In addition to the fNIRS testing, a Mood Portraits® study as described in Example 2 was also conducted on a large number of fragrance compositions.

For the present invention, the results of the Mood Portraits® study were analysed with regard to a happy/uplifted mood. Specifically, the selection frequency of pictures associated with happiness and the grade of association of the respective pictures with a positive happy mood (some pictures are very strongly associated with happiness, whereas it is only one association among several equally strong ones for other pictures) were taken into account.

A comparison of several dozen fragrance compositions showed that most of them have a very similar effect on happiness; but a few fragrance compositions are able to significantly evoke or not evoke a happy mood.

FIG. 2 shows the results for some of the fragrance compositions that were tested, namely for Compositions I (according to the invention) and N (comparative example) of Example 3, and of Compositions Q through W, which were not previously described in this disclosure.

More precisely, FIG. 2 shows the odds ratio for a happy/uplifted mood, indicating for each fragrance composition if it evokes a happy mood more or less than the other compositions. The odds ratios are shown as dots. If the 95% confidence interval for the odds ratio for a fragrance composition is entirely above the Significance Line of 1.0, then said fragrance composition significantly evokes a happier mood and is marked in FIG. 2 by an arrow; if the 95% confidence interval for a fragrance composition is entirely below the Significance Line of 1.0, then said fragrance composition significantly evokes a less happy mood.

Thus, as can be seen from FIG. 2, Composition I, which is a fragrance composition according to the present invention, is able to evoke a happy/uplifted mood significantly more than all the other fragrance compositions. Compositions N and Q through W essentially lie on the Significance Line.

Thus, the Mood Portraits® results confirm that Composition I, which has been found to be happy in the fNIRS study and which also complies with the formulation guidelines of the present invention, significantly evokes more happiness compared to a large majority of other fragrance compositions.

1. A method of assessing the ability of a test fragrance ingredient or a test fragrance composition to improve the happiness state of a human subject, comprising the steps of:
a) measuring a base happiness state of one or more human test subject(s);
b) providing the test fragrance ingredient or the test fragrance composition to the human test subject(s) for smelling;
c) measuring a resulting happiness state of the human test subject(s); and
d) determining a difference between the resulting happiness state and the base happiness state for the human test subject(s);
wherein the base happiness state and the resulting happiness state are measured by functional Near Infrared Spectroscopy (fNIRS) of the human test subject(s)′ left brain hemisphere, right brain hemisphere, and full brain;
wherein the test fragrance ingredient or the test fragrance composition is able to improve the happiness state of the human subject if at least one of Criterion A and Criterion B is met,
wherein Criterion A requires that at least three out of the following six conditions A1 through A6 are met:
A1. Deoxy Hb for the full brain shows a statistically significant decrease after 30 seconds of smelling;
A2. Deoxy Hb for the right brain hemisphere shows a statistically significant decrease after 30 seconds of smelling;
A3. Total Hb for the full brain shows a statistically significant decrease after 30 seconds of smelling;
A4. Total Hb for the right brain hemisphere shows a statistically significant decrease after 30 seconds of smelling;
A5. Deoxy Hb for the right brain hemisphere shows a statistically significant increase after 0-10 seconds of smelling;
A6. Oxy Hb for the right brain hemisphere shows a statistically significant increase after 5-10 seconds of smelling;
wherein Deoxy Hb is the amount of deoxygenated haemoglobin measured, wherein Total Hb is the amount of total haemoglobin measured, and wherein Oxy Hb is the amount of oxygenated haemoglobin measured; and
wherein Criterion B requires that at least five out of the following ten conditions B1 through B10 are met:
B1. Channel 3 shows a statistically significant increase of Deoxy Hb after 30 seconds of smelling;
B2. Channel 20 shows a statistically significant increase of Deoxy Hb after 30 seconds of smelling;
B3. Channel 12 shows a statistically significant decrease of Total Hb after 30 seconds of smelling;
B4. Channel 5 shows a statistically significant increase of Oxy Hb after 0-5 seconds of smelling;
B5. Channel 4 shows a statistically significant increase of Total Hb after 0-5 seconds of smelling;
B6. Channel 5 shows a statistically significant increase of Total Hb after 0-5 seconds of smelling;
B7. Channel 11 shows a statistically significant increase of Oxy Hb after 0-10 seconds of smelling;
B8. Channel 8 shows a statistically significant decrease of Deoxy Hb after 5-10 seconds of smelling;
B9. Channel 10 shows a statistically significant decrease of Deoxy Hb after 5-10 seconds of smelling;
B10. Channel 18 shows a statistically significant increase of Oxy Hb after 5-10 seconds of smelling;
wherein Channels 1 to 8 and 11 are located in the left brain hemisphere, Channels 9 and 12 are located on the midline, and Channels 10 and 13 to 20 are located in the right brain hemisphere.

2. The method of claim 1, wherein both Criterion A and Criterion B are met.

3. The method of claim 1, wherein further Criterion C is met, wherein Criterion C requires that at least ten, more preferably at least twelve, and most preferably at least 15, out of the following 20 conditions B1 through B10 and C1 through C10 are met:
B1. Channel 3 shows a statistically significant increase of Deoxy Hb after 30 seconds of smelling;
B2. Channel 20 shows a statistically significant increase of Deoxy Hb after 30 seconds of smelling;
B3. Channel 12 shows a statistically significant decrease of Total Hb after 30 seconds of smelling;
B4. Channel 5 shows a statistically significant increase of Oxy Hb after 0-5 seconds of smelling;
B5. Channel 4 shows a statistically significant increase of Total Hb after 0-5 seconds of smelling;
B6. Channel 5 shows a statistically significant increase of Total Hb after 0-5 seconds of smelling;
B7. Channel 11 shows a statistically significant increase of Oxy Hb after 0-10 seconds of smelling;
B8. Channel 8 shows a statistically significant decrease of Deoxy Hb after 5-10 seconds of smelling;
B9. Channel 10 shows a statistically significant decrease of Deoxy Hb after 5-10 seconds of smelling;
B10. Channel 18 shows a statistically significant increase of Oxy Hb after 5-10 seconds of smelling;
C1. Channel 4 shows a statistically significant increase of Oxy Hb after 30 seconds of smelling;
C2. Channel 4 shows a statistically significant increase of Total Hb after 30 seconds of smelling;
C3. Channel 19 shows a statistically significant increase of Total Hb after 30 seconds of smelling;
C4. Channel 2 shows a statistically significant decrease of Deoxy Hb after 0-5 seconds of smelling;
C5. Channel 8 shows a statistically significant decrease of Deoxy Hb after 0-5 seconds of smelling;
C6. Channel 6 shows a statistically significant increase of Oxy Hb after 0-5 seconds of smelling;
C7. Channel 12 shows a statistically significant decrease of Oxy Hb after 0-5 seconds of smelling;
C8. Channel 13 shows a statistically significant increase of Oxy Hb after 0-5 seconds of smelling;
C9. Channel 16 shows a statistically significant increase of Oxy Hb after 0-5 seconds of smelling;
C10. Channel 6 shows a statistically significant increase of Oxy Hb after 5-10 seconds of smelling;
wherein Channels 1 to 8 and 11 are located in the left brain hemisphere, Channels 9 and 12 are located on the midline, and Channels 10 and 13 to 20 are located in the right brain hemisphere.

4. The method of claim 3, wherein further Criterion D is met, wherein Criterion D requires:
that at least 16, more preferably at least 17, and most preferably at least 18, out of the 20 conditions B1 through B10 and C1 through C10 are met if zero or one out of the conditions A1 through A6 are met;
that at least 12, more preferably at least 13, and most preferably at least 14, out of the 20 conditions B1 through B10 and C1 through C10 are met if two or three out of the conditions A1 through A6 are met; and
that at least 8, more preferably at least 9, and most preferably at least 10, out of the 20 conditions B1 through B10 and C1 through C10 are met if four, five or six out of the conditions A1 through A6 are met.

5. The method of claim 1, wherein at least one of Criterion E1 and Criterion E2 are met, wherein
Criterion E1 requires that at least four, more preferably at least five, and most preferably all six out of the conditions A1 through A6 are met;
Criterion E2 requires that at least six, more preferably at least seven, and most preferably at least eight out of the ten conditions B1 through B10 are met.

6. A method of creating a fragrance composition having an effect of improving the happiness of a human subject, comprising the steps of:
(i) creating a test fragrance composition;
(ii) assessing the ability of the test fragrance composition to improve the happiness state of a human subject according to the method of any one of claim 1; and
(iii) adjusting, if necessary, the test fragrance composition by adding and/or removing at least one fragrance ingredient and/or increasing and/or reducing the concentration of at least one fragrance ingredient until the fragrance composition is found to improve the happiness state of the human subject.

7. The method of claim 6, wherein, in step (iii), at least one HMP fragrance ingredient is added to the test fragrance composition and/or at least one HMR fragrance ingredient is added to the test fragrance composition and/or at least one HMI fragrance ingredient is added to the test fragrance composition and/or at least one RMP fragrance ingredient is removed from the test fragrance composition and/or at least one IMPU fragrance ingredient is removed from the test fragrance composition and/or the concentration of at least one HMP, HMR and/or HMI fragrance ingredient is increased and/or the concentration of at least one RMP and/or IMPU fragrance ingredient is reduced, wherein
the HMP fragrance ingredients are selected from the group consisting of fruity-candied fruit ingredients (excluding damascone alpha), fruity-strawberry ingredients, fruity-raspberry ingredients, fruity-pineapple ingredients, grapefruit oil, 6,6-dimethoxy-2,5,5-trimethylhex-2-ene (methyl pamplemousse), hexenyl-3-salicylate, ylang ylang oil, ethyl 3-oxobutanoate (ethyl acetoacetate), 5-hexyloxolan-2-one (gamma decalactone), 5-octyloxolan-2-one (dodecalatone gamma), 2,2,5-trimethyl-5-pentylcyclopentan-1-one (veloutone), hexyl acetate, cassis base, 1-phenylethyl acetate (styrallyl acetate), (E)-4-methyldec-3-en-5-ol (undecavertol), 2-ethyl-3-hydroxypyran-4-one (ethyl maltol), 8-methyl-1,5-benzodioxepin-3-one (calone), 1-[(1R,2R,5S,7R)-2,6,6,8-tetramethyl-9-tricyclo[5.3.1.01,5]undec-8-enyl]ethanone (methyl cedryl ketone or vertofix coeur), and mixtures thereof;
the HMR fragrance ingredients are selected from the group consisting of lemon oil, (E)-3,7-dimethylnona-1,6-dien-3-ol (ethyl linalool), benzyl acetate, 3-methyl-2-[(Z)-pent-2-enyl]cyclopent-2-en-1-one (jasmone-cis), 2-(2′-methylpropyl)-4-hydroxy-4-methyl-tetrahydropyran (Florosa), (E)-1-(2,6,6-trimethylcyclohex-2-en-1-yl)hepta-1,6-dien-3-one (cetone V), N-1-(2,6,6-trimethyl-1-cyclohex-2-enyl)pent-1-en-3-one (Isoraldeine), 3,7-dimethyloct-6-en-1-ol (citronellol), (E)-3,7-dimethylocta-2,6-dien-1-ol (geraniol), geranium oil, 4-(4-hydroxy-4-methylpentyl)cyclohex-3-enecarbaldehyde (Lyral, Cyclohexal), 5-heptyldihydrofuran-2(3H)-one (peach pure, undecalactone gamma), 1,4-dioxacycloheptadecane-5,17-dione (ethylene brassylate), mixtures of cyclohexa-decanolide and cyclopentadecanone (Silvanone), (5E)-3-methylcyclopentadec-5-en-1-one (Muscenone), (E)-2-methoxy-4-(prop-1-en-1-yl)phenol (isoeugenol), and mixtures thereof;
the HMI fragrance ingredients are selected from the group consisting of citrus-orange ingredients, citrus-mandarin ingredients, 1-(2-tert-butylcyclohexyl)oxybutan-2-ol (amber core), (3aR,5aS,9aS,9bR)-3a,6,6,9a-tetramethyl-2,4,5,5a, 7,8,9,9b-octahydro-1H-benzo[e][1]benzofuran (ambrofix or ambroxan), 1,5,5,9-tetramethyl-13-oxatricyclo (8.3.0.0.(4.9))tridecane (cetalox or fixambrene), 2,6-dimethyloct-7-en-2-ol (dihydro-myrcenol), 3-(1,3-benzodioxol-5-yl)-2-methylpropanal (helional or tropional), (2-tert-butylcyclohexyl) acetate (agrumex), 1-(2,6,6-trimethyl-1-cyclohex-2-enyl)but-3-en-1-one (damascone alpha), 2,4-dimethyl-3-cyclohexene-1-carbaldehyde (cyclal C or tricyclal or ligustral), 4-allyl-2-methoxyphenol (eugenol), and mixtures thereof;
the RMP fragrance ingredients are selected from the group consisting of 2-(4-methylcyclohex-3-en-1-yl)propan-2-ol (terpineol), hexyl 2-hydroxybenzoate (hexyl salicylate), jasmin oil, 7-hydroxy-3,7-dimethyloctanal (hydroxycitronellal), 3-methyl-5-phenylpentanol (Mefrosol), 2-(phenoxy)ethyl 2-methylpropanoate (phenoxyethyl iso-butyrate), (12E)-1-oxacyclohexadec-12-en-2-one (Habanolide), 4-methoxybenzalde-hyde (aubepine para cresol, anisic aldehyde), benzoin resinoids, 3-ethoxy-4-hydroxybenzaldehyde (ethyl vanillin), 4-hydroxy-3-methoxybenzaldehyde (vanillin), 2-ethyl-4(2′,2′,3′-trimethylcyclopent-3-enyl)but-enol (Bangalol or Radjanol), mixtures of 2-methyl-1-phenylpropan-2-yl butanoate and (phenoxy)ethyl 2-methylpropanoate (Prunella), and mixtures thereof; and
the IMPU fragrance ingredients are selected from the group consisting of aromatic-eucalyptus ingredients, aromatic-mint ingredients, aromatic-rosemary ingredients, citrus-lime in-gredients, spicy-pepper ingredients, citrus-floral/lemon ingredients, lavandin oil, patchouli oil, clary sage oil, orange flower oil, guaiacwood oil, oakmoss oil, litsea cubeba oil, citral, benzyl 2-hydroxybenzoate (benzyl salicylate), 2-methyl-3-(4-(1-methylethyl)phenyl)propanal (cyclamen aldehyde), 3-(4-ethylphenyl)-2,2-dimethylpropanal (floralozone), prop-2-enyl 2-(3-methylbutoxy)acetate (allyl amyl glycolate), prop-2-enyl 2-cyclohexyloxyacetate (cyclogalbanate), 4-methyl-2-(2-methylprop-1-en-1-yl)tetrahydro-2H-pyran (rose oxide), 1-[(2Z,5Z,9Z)-2,6,10-trimethylcyclododeca-2,5,9-trien-1-yl]ethanone (trimofix O or cyclisone), methyl 2,4-dihydroxy-3,6-dimethylbenzoate (Evernyl or Everniate), 2,6-dimethylheptan-2-ol (dimetol), 3,7-dimethylocta-1,6-dien-3-yl acetate (linalyl acetate) fir balsam oil, pine needle base, and mixtures thereof.

8. A fragrance composition for improving the happiness state of a human subject, the fragrance composition comprising at least 75%, preferably at least 85%, of fragrance ingredients drawn from the following groups:
a) at least about 5% by weight in total of at least three HMP fragrance ingredients;
b) optionally up to about 95% by weight in total of HMR, HMI, IMPU, RMP b) and/or GEN fragrance ingredients, provided the following conditions are met:
(b1) HMPs+HMRs≥IMPUs
(b2) HMPs+HMIs≥RMPs
(b3) HMRs+HMIs+HMPs+GENs≥65%;
(b4) HMPs/(HMPs+RMPs+IMPUs)≥0.35
(b5) HMPs/(HMPs+RMPs+IMPUs)+(100-TOTAL)≥0.2
wherein
(i) all percentages are based on total weight of the fragrance ingredients constituting the fragrance composition;
(ii) HMPs indicates the sum of percentages of HMP fragrance ingredients; HMRs indicates the sum of percentages of HMR fragrance ingredients; HMIs indicates the sum of percentages of HMI fragrance ingredients; IMPUs indicates the sum of percentages of IMPU fragrance ingredients; RMPs indicates the sum of percentages of RMP fragrance ingredients; GENs indicates the sum of percentages of GEN fragrance ingredients; and TOTAL indicates the sum of HMPs, HMRs, HMIs, IMPU, RMPs, and GENs; provided that low odour or no odour solvents, diluents and other vehicles are excluded from the calculation of these sums;
(iii) the symbol ≥ indicates at least equal to;
(iv) the HMP fragrance ingredients are selected from the group consisting of fruity-candied fruit ingredients (excluding damascone alpha), fruity-strawberry ingredients, fruity-raspberry ingredients, fruity-pineapple ingredients, grapefruit oil, 6,6-dimethoxy-2,5,5-trimethylhex-2-ene (methyl pamplemousse), hexenyl-3-salicylate, ylang ylang oil, ethyl 3-oxobutanoate (ethyl acetoacetate), 5-hexyloxolan-2-one (gamma decalactone), 5-octyloxolan-2-one (dodecalatone gamma), 2,2,5-trimethyl-5-pentylcyclopentan-1-one (veloutone), hexyl acetate, cassis base, 1-phenylethyl acetate (styrallyl acetate), (E)-4-methyldec-3-en-5-ol (undecavertol), 2-ethyl-3-hydroxypyran-4-one (ethyl maltol), 8-methyl-1,5-benzodioxepin-3-one (calone), 1-[(1R,2R,5S,7R)-2,6,6,8-tetramethyl-9-tricyclo [5.3.1.01,5]undec-8-enyl]ethanone (methyl cedryl ketone or vertofix coeur), and mixtures thereof;
(v) the HMR fragrance ingredients are selected from the group consisting of lemon oil, (E)-3,7-dimethylnona-1,6-dien-3-ol (ethyl linalool), benzyl acetate, 3-methyl-2-[(Z)-pent-2-enyl]cyclopent-2-en-1-one (jasmone-cis), 2-(2′-methylpropyl)-4-hydroxy-4-methyltetrahydropyran (Florosa), (E)-1-(2,6,6-trimethylcyclohex-2-en-1-yl)hepta-1,6-dien-3-one (cetone V), N-1-(2,6,6-trimethyl-1-cyclohex-2-enyl)pent-1-en-3-one (Isoraldeine), 3,7-dimethyloct-6-en-1-ol (citronellol), (E)-3,7-dimethylocta-2,6-dien-1-ol (geraniol), geranium oil, 4-(4-hydroxy-4-methylpentyl)cyclohex-3-enecarbaldehyde (Lyral, Cyclohexal), 5-heptyldihydrofuran-2(3H)-one (peach pure, undecalactone gamma), 1,4-dioxacycloheptadecane-5,17-dione (ethylene brassylate), mixtures of cyclohexadecanolide and cyclopentadecanone (Silvanone), (5E)-3-methylcyclopentadec-5-en-1-one (Muscenone), (E)-2-methoxy-4-(prop-1-en-1-yl)phenol (isoeugenol), and mixtures thereof;
(vi) the HMI fragrance ingredients are selected from the group consisting of citrus-orange ingredients, citrus-mandarin ingredients, 1-(2-tert-butylcyclohexyl) oxybutan-2-ol (amber core), (3aR,5aS,9aS,9bR)-3a,6,6,9a-tetramethyl-2,4,5,5a, 7,8,9,9b-octahydro-1H-benzo[e][1]benzofuran (ambrofix or ambroxan), 5,5,9-tetra-methyl-13-oxatricyclo(8.3.0.0.(4.9))tridecane (cetalox or fixambrene), 2,6-dimethyl-oct-7-en-2-ol (dihydromyrcenol), 3-(1,3-benzodioxol-5-yl)-2-methylpropanal (helional or tropional), (2-tert-butylcyclohexyl) acetate (agrumex), 1-(2,6,6-trimethyl-1-cyclohex-2-enyl)but-3-en-1-one (damascone alpha), 2,4-dimethyl-3-cyclohexene-1-carbaldehyde (cyclal C or tricyclal or ligustral), 4-allyl-2-methoxyphenol (eugenol), and mixtures thereof;
(vii) the IMPU fragrance ingredients are selected from the group consisting of aromatic-eucalyptus ingredients, aromatic-mint ingredients, aromatic-rosemary ingredients, citrus-lime in-gredients, spicy-pepper ingredients, citrus-floral/lemon ingredients, lavandin oil, patchouli oil, clary sage oil, orange flower oil, guaiacwood oil, oakmoss oil, litsea cubeba oil, citral, benzyl 2-hydroxybenzoate (benzyl salicylate), 2-methyl-3-(4-(1-methylethyl)phenyl)propanal (cyclamen aldehyde), 3-(4-ethylphenyl)-2,2-dimethylpropanal (floralozone), prop-2-enyl 2-(3-methylbutoxy)acetate (allyl amyl glycolate), prop-2-enyl 2-cyclohexyloxyacetate (cyclogalbanate), 4-methyl-2-(2-methylprop-1-en-1-yl)tetrahydro-2H-pyran (rose oxide), 1-[(2Z,5Z,9Z)-2,6,10-trimethylcyclododeca-2,5,9-trien-1-yl]ethanone (trimofix O or cyclisone), methyl 2,4-dihydroxy-3,6-dimethylbenzoate (Evernyl or Everniate), 2,6-dimethylheptan-2-ol (dimetol), 3,7-dimethylocta-1,6-dien-3-yl acetate (linalyl acetate) fir balsam oil, pine needle base, and mixtures thereof;
(viii) the RMP fragrance ingredients are selected from the group consisting of 2-(4-methylcyclohex-3-en-1-yl)propan-2-ol (terpineol), hexyl 2-hydroxybenzoate (hexyl salicylate), jasmin oil, 7-hydroxy-3,7-dimethyloctanal (hydroxycitronellal), 3-methyl-5-phenylpentanol (Mefrosol), 2-(phenoxy)ethyl 2-methylpropanoate (phenoxyethyl isobutyrate), (12E)-1-oxacyclohexadec-12-en-2-one (Habanolide), 4-methoxy-benzaldehyde (aubepine para cresol, anisic aldehyde), benzoin resinoids, 3-ethoxy-4-hydroxybenzaldehyde (ethyl vanillin), 4-hydroxy-3-methoxybenzaldehyde (vanillin), 2-ethyl-4(2′,2′,3′-trimethylcyclopent-3-enyl)but-enol (Bangalol or Radjanol), mixtures of 2-methyl-1-phenylpropan-2-yl butanoate and (phenoxy)ethyl 2-methylpropanoate (Prunella), and mixtures thereof; and
(ix) the GEN fragrance ingredients are selected from the group consisting of 3,7-dimethylocta-1,6-dien-3-ol (linalool), methyl 3-oxo-2-pentylcyclopentaneacetate (methyl dihydro-jasmonate, cepionate, hedione), hexyl cinnamic aldehyde, 3-(4-(1,1-dimethylethyl)phenyl-2-methylpropanal (Lilial), (E)-4-(2,6,6-trimethylcyclohex-1-en-1-yl)but-3-en-2-one (ionone beta), 2-phenylethyl alcohol, 1-(2,3,8,8-tetramethyl-1,3,4,5,6,7-hexahydronaphthalen-2-yl)ethanone (sylvamber or iso e super or iso gamma super), 1,15-pentadecanolide (Thibetolide), (isocamphyl-5)cyclohexanol (sandela), (3R,3aS,6R,7R,8aS)-octahydro-6-methoxy-3,6,8,8-tetramethyl-1H-3a, 7-methanoazulene (cedryl methyl ether), and mixtures thereof.

9. The fragrance composition of claim 8, comprising at least about 10%, more preferably at least about 15%, by weight in total of HMP fragrance ingredients.

10. The fragrance composition of claim 8, comprising at least four HMP fragrance ingredients, more preferably at least five HMP fragrance ingredients.

11. The fragrance composition of claim 8, comprising at least one HMP, HMI and/or HMR fragrance ingredient selected from one or more of the following groups:
one or more fruity-candied fruit ingredients selected from the group consisting of (E)-1-(2,6,6-trimethylcyclohexa-1,3-dien-1-yl)but-2-en-1-one (damascenone), dimethyl benzyl carbinyl butyrate, (E)-1-(2,6,6-trimethyl-1-cyclohexenyl)but-2-en-1-one (damascone beta), (E)-1-(2,2-dimethyl-6-methylidenecyclohexyl)but-2-en-1-one (damascone gamma), (E)-1-(2,6,6-trimethyl-1-cyclohex-3-enyl)but-2-en-1-one (damascone delta), tagetes oil, ethyl 2-ethyl-6,6-dimethylcyclohex-2-ene-1-carboxylate (givescone), ethyl 2,6,6-trimethylcyclohexa-1,3-diene-1-carboxylate (ethyl safranate), ethyl 2,6,6-trimethylcyclohexa-1,3-diene-1-carboxylate (cristalon), (2E,5Z)-5,6,7-trimethylocta-2,5-dien-4-one (pomerose), (3□,4□,7□,7□)-octahydro-4,7-methano-3aH-indene-3a-carboxylic acid ethyl ester (fruitate), and mixtures thereof;
one or more fruity-strawberry ingredients selected from the group consisting of benzyl cinnamate, ethyl 3-methyl-3-phenyloxirane-2-carboxylate (strawberry pure), ethyl butanoate (ethyl butyrate), ethyl 2-methylpropionate (ethyl isobutyrate), ethyl cyclohexanecarboxylate (esterly), ethyl cinnamate, methyl cinnamate, benzyl cinnamate, ethyl phenyl glycidate, phenyl ethyl butyrate, benzyl butyrate, ethyl isovalerate, phenyl ethyl isovalerate, 2-octene-4-one, and mixtures thereof;
one or more fruity-raspberry ingredients selected from the group consisting of 4-(4-hydroxyphenyl)butan-2-one (raspberry ketone), methyoxy phenyl butanone, ethyl 6-acetyloxyhexanoate (berryflor), and mixtures thereof;
Ione or more fruity-pineapple ingredients selected from the group consisting of prop-2-enyl 3-cyclohexylpropanoate (allyl cyclohexyl propionate), prop-2-enyl heptanoate (allyl oenanthate), ethyl octanoate (ethyl oenanthate), 3-methylbutyl octanoate (isoamyl caproate), methyl hexanoate, ethyl hexanoate, pentyl hexanoate (amyl caproate), phenyl ethyl isobuyrate, allyl propionate, methyl octanoate, and mixtures thereof;
one or more citrus-orange ingredients selected from the group consisting of orange oil, orange terpenes, orange aldehyde, and mixtures thereof; and/or
one or more citrus-mandarin ingredients selected from the group consisting of mandarin oil, tangerine oil, (E)-6,10-dimethylundeca-5,9-dien-2-yl acetate (tangerinol), methyl 2-methylaminobenzoate (dimethyl anthranilate), octanol-3, and mixtures thereof.

12. The fragrance composition of claim 8, wherein HMRs+HMIs+HMPs+GENs≥70%, more preferably HMRs+HMIs+HMPs+GENs≥75%, and most preferably HMRs+HMIs+HMPs+GENs≥80%.

13. The fragrance composition of claim 8, wherein HMPs/(HMPs+RMPs+IMPUs)≥0.40, more preferably HMPs/(HMPs+RMPs+IMPUs)≥0.45, and most preferably HMPs/(HMPs+RMPs+IMPUs)≥0.50.

14. A consumer product comprising the fragrance composition of claim 8.

15. A method of improving the happiness state of a human subject, comprising the step of providing an effective amount of the fragrance composition of claim 8 to the human subject.

16. A method of using a fragrance ingredient in improving the happiness state of a human subject, wherein the fragrance ingredient is selected from the group consisting of:
one or more fruity-candied fruit ingredients selected from the group consisting of dimethyl benzyl carbinyl butyrate, (E)-1-(2,6,6-trimethyl-1-cyclohexenyl)but-2-en-1-one (damascone beta), (E)-1-(2,2-dimethyl-6-methylidenecyclohexyl)but-2-en-1-one (damascone gamma), (E)-1-(2,6,6-trimethyl-1-cyclohex-3-enyl)but-2-en-1-one (damascone delta), tagetes oil, ethyl 2-ethyl-6,6-dimethylcyclohex-2-ene-1-carboxylate (givescone), ethyl 2,6,6-trimethylcyclohexa-1,3-diene-1-carboxylate (ethyl safranate), ethyl 2,6,6-trimethylcyclohexa-1,3-diene-1-carboxylate (cristalon), (2E,5Z)-5,6,7-trimethylocta-2,5-dien-4-one (pomerose), (3□,4□,7□,7□)-octahydro-4,7-methano-3aH-indene-3a-carboxylic acid ethyl ester (fruitate), and mixtures thereof;
one or more fruity-strawberry ingredients selected from the group consisting of benzyl cinnamate, ethyl butanoate (ethyl butyrate), ethyl 2-methylpropionate (ethyl isobutyrate), ethyl cyclohexanecarboxylate (esterly), ethyl cinnamate, methyl cinnamate, benzyl cinnamate, ethyl phenyl glycidate, phenyl ethyl butyrate, benzyl butyrate, ethyl isovalerate, phenyl ethyl isovalerate, 2-octene-4-one, and mixtures thereof;
one or more fruity-raspberry ingredients selected from the group consisting methyoxy phenyl butanone, ethyl 6-acetyloxyhexanoate (berryflor), and mixtures thereof;
one or more fruity-pineapple ingredients selected from the group consisting of ethyl octanoate (ethyl oenanthate), 3-methylbutyl octanoate (isoamyl caproate), methyl hexanoate, ethyl hexanoate, pentyl hexanoate (amyl caproate), phenyl ethyl isobuyrate, allyl propionate, methyl octanoate, and mixtures thereof;
one or more citrus-orange ingredients selected from the group consisting of orange aldehyde, and mixtures thereof; and/or
one or more citrus-mandarin ingredients selected from the group consisting of tangerine oil, (E)-6,10-dimethylundeca-5,9-dien-2-yl acetate (tangerinol), methyl 2-methylaminobenzoate (dimethyl anthranilate), octanol-3, and mixtures thereof.
